(19) United States
(12) Patent Application Publication
(10) Pub. No.: US 2013/0281513 A1
(43) Pub. Date: Oct. 24, 2013

(54) siRNA FOR INHIBITION OF Hif1alpha EXPRESSION AND ANTICANCER COMPOSITION CONTAINING THE SAME

(71) Applicants: Sun-Ok Kim, Daejeon (KR); Sang-Hee Kim, Daejeon (KR); Eun-Ah Cho, Daejeon (KR); Chang-Hoon In, Cheongju-si (KR)

(72) Inventors: Sun-Ok Kim, Daejeon (KR); Sang-Hee Kim, Daejeon (KR); Eun-Ah Cho, Daejeon (KR); Chang-Hoon In, Cheongju-si (KR)

(73) Assignee: Samyang Biopharmaceuticals Corporation, Seoul (KR)

(21) Appl. No.: 13/993,765

(22) PCT Filed: Dec. 29, 2011
(86) PCT No.: PCT/KR11/10318

§ 371 Date: Jun. 13, 2013

(30) Foreign Application Priority Data
Dec. 30, 2010 (KR) 10-2010-0139391

(51) Int. Cl. A61K 45/06; A61K 31/713

(52) U.S. Cl. CPC A61K 45/06; A61K 31/713
USPC 514/44 A; 536/24.5; 435/320.1; 435/375

(57) ABSTRACT

Disclosed are small interfering RNA (siRNA) that complementarily binds to a base sequence of Hif1α mRNA transcript, thereby inhibiting expression of Hif1α without inducing immune responses, and a use of the siRNA for prevention and/or treatment of cancer. Since Hif1α is commonly overexpressed in almost all cancer cells, the siRNA that complementarily binds to Hif1α-encoding mRNA may inhibit expression of Hif1α through RNA-mediated interference (RNAi), thereby inhibiting proliferation and metastasis of cancer cells, and thus, the siRNA may be useful as an anticancer agent.

### BACKGROUND OF THE INVENTION

(a) Field of the Invention

The present invention relates to a small interfering RNA (siRNA) that complementary binds to a base sequence of Hif1α mRNA transcript, thereby inhibiting expression of Hif1α without activating immune responses, and a use of the siRNA for prevention and/or treatment of cancer.

(b) Description of the Related Art

Hif1 (Hypoxia inducible factor 1) is a heterodimeric transcription factor consisting of Hif1α subunit controlling the substantial activity of Hif1αnd Hif-1β subunit functioning as a nuclear transporter. Both subunits are members of the basic-helix-loop-helix-PAS (PER-ARNT-SIM) super-family. Under normoxia, Hif1α is rapidly degraded. Degradation occurs when the VHL (von Hippel Lindau, a recognition component of the E3ubiquitin ligase system, binds hydroxylated proline (Pro594 and Pro402) residues of ODD (oxygen-degradation domain). However, under hypoxia (oxygen rate 5% or less) which is commonly generated phenomenon in various solid cancers, such hydroxylation is inhibited, and thus, Hif1α is not degraded, and moves from cytoplasm to nucleus in a dimer form and binds to HRE (hypoxia response element), thereby inducing expression of genes involved in angiogenesis, glycolysis, cell growth, and differentiation (Veronica A. et al., Cancer Research, 66(12), 6264-70, 2006; Semenza G L. et al., Nature Review Cancer 3, 721-32, 2003). The regulation of HIF-1 activity occurs at multiple levels.

Since the regulation of Hif1α activity occurs at multiple levels, it is considered to be the best way to fundamentally inhibit Hif1α expression using siRNA targeting Hif1α mRNA rather than to target the pathways of these mechanisms.

Recently, it has been revealed that the ribonucleic acid-mediated interference (RNAi) contributes to development of drug lead-candidate by exhibiting sequence specific gene silencing even for otherwise non-druggable targets with the existing technologies. Therefore, RNAi has been considered as a technology capable of suggesting solutions to the problems of limited targets and non-specificity in synthetic drugs, and overcoming limitations of chemical synthetic drugs, and thus, a lot of studies on the use thereof in development of medicines for various diseases that is hard to be treated by the existing technologies, in particular cancer, are actively progressed.

Ribonucleic acid mediated interference (RNAi) is a phenomenon that ribonucleic acid consisting of 21-25 bases and having a double helix structure complementarily binds to mRNA transcript of a target gene and degrades the transcript, thereby inhibiting expression of the target gene (Novina & Sharp, Nature, 430:161-164, 2004).

However, it was found out that siRNA (small interfering RNA) triggers innate immune responses, and also induces non-specific RNAi effect more frequently than expected.

It has been reported that in mammal cells, long double stranded siRNA may induce a deleterious interferon response; short double stranded siRNA may also induce an initial interferon response deleterious to the human body or cells; and many siRNAs have been known to induce higher non-specific RNAi effect than expected (Kleirman et al. Nature, 452:591-7, 2008).

Although there has been an attempt to develop siRNA anticancer drugs targeting Hif1α which plays an important role in the progression of cancer, so far the outcome is insignificant. Gene inhibition effect of individual sequence of siRNA has not been suggested, and particularly, immune activity has not been considered.

Although siRNA shows great promise as a novel medicine due to the advantages such as high activity, excellent target specificity, and the like, it has several obstacles to overcome for therapeutic development, such as low blood stability because it may be degraded by nuclease in blood, a poor ability to pass through cell membrane due to negative charge, short half life in blood due to rapid excretion, whereby its limited tissue distribution, and induction of off-target effect capable of affecting on regulation pathway of other genes.

### SUMMARY OF THE INVENTION

Accordingly, the inventors developed siRNA that has high sequence specificity and thus specifically binds to transcript of a target gene to increase RNAi activity, and does not induce any immune toxicity, to complete the invention.

One embodiment provides a siRNA that complementarily binds to Hif1α mRNA transcript, thereby specifically inhibiting synthesis and/or expression of Hif1α.

Another embodiment provides an expression vector for expressing the siRNA.

Another embodiment provides a pharmaceutical composition for inhibiting synthesis and/or expression of Hif1α, comprising the siRNA or the siRNA expression vector as an active ingredient.

Another embodiment provides an anticancer composition comprising the siRNA or the siRNA expression vector as an active ingredient.

Another embodiment provides a method of inhibiting synthesis and/or expression of Hif1α, comprising the step of contacting the siRNA or siRNA expression vector with Hif1α-expressing cells, and a use of the siRNA or siRNA expression vector for inhibiting synthesis and/or expression of Hif1α in Hif1α-expressing cells.

Another embodiment provides a method of inhibiting growth of cancer cells comprising contacting the siRNA or siRNA expression vector with Hif1α-expressing cancer cells, and a use of the siRNA or siRNA expression vector for inhibiting cell growth in Hif1α-expressing cancer cells.

Still another embodiment provides a method of preventing and/or treating a cancer, comprising the step of administering the siRNA or siRNA expression vector in a therapeutically effective amount to a patient in need thereof, and use of the siRNA or siRNA expression vector for prevention and/or treatment of a cancer.

### DETAILED DESCRIPTION OF THE EMBODIMENTS

The present invention provides siRNA that complementarily binds to Hif1αmRNA transcript base sequence, thereby inhibiting synthesis and/or expression of Hif1α in a cell, a pharmaceutical composition comprising the same, and a use thereof.

According to one aspect of the present invention, provided is siRNA for specifically inhibiting synthesis and/or expression of Hif1α. According to another aspect, provided is a pharmaceutical composition for inhibiting synthesis and/or expression of Hif1α, comprising the siRNA specifically inhibiting synthesis and/or expression of Hif1α as an active ingredient. According to yet another aspect, provided is an agent for inhibiting cancer cell growth, or a pharmaceutical composition (anticancer composition) for prevention and/or treatment of a cancer, comprising the siRNA specifically inhibiting synthesis and/or expression of Hif1α as an active ingredient.

The present invention relates to a technology of inhibiting expression of Hif1α mRNA of mammals including human, an alternative splice form thereof, and/or Hif1α gene of the same line, which may be achieved by administering a specific amount of the siRNA of the present invention to a patient, to reduce the target mRNA expression.

Hereinafter, the present invention will be described in detail.

The Hif1α may be originated from mammals, preferably human, or it may be Hif1α of the same line as human and an alternative splice form thereof. The term ‘same line as human’ refers to mammals having genes or mRNA with 80% or more sequence identity to human Hif1α genes or mRNA originated therefrom, and specifically, it may include human, primates, rodents, and the like.

According to one embodiment, cDNA sequence of a sense strand corresponding to Hif1α-encoding mRNA may be as shown in SEQ ID NO 1.

The siRNA according to the present invention may target a region consisting of consecutive 15 to 25 bp, preferably consecutive 18 to 22 bp in mRNA or cDNA of Hif1α (for example, SEQ ID NO 1), specifically the mRNA region corresponding to at least one base sequence selected from the group consisting of SEQ ID NOs: 2, 3 and 5 to 14 (base sequence of cDNA). Preferable target regions on cDNA are summarized in the following Table 1. Thus, according to one embodiment of the invention, provided is siRNA for targeting the mRNA region corresponding to at least one base sequence selected from the group consisting of SEQ ID NOs: 2, 3 and 5 to 14 in the Hif1α cDNA of SEQ ID NO: 1. For example, provided is siRNA for targeting the mRNA region corresponding to base sequence selected from the group consisting of SEQ ID NOs: 6, 10, and 12.

TABLE 1
Seventeen (17) Target regions on Hif1α
cDNA(SEQ ID NO: 1),

| | | | Starting |
|---|---|---|---|
| | SEQ | | nucleotide |
| Sequence | ID | sequence | in Hif1α |
| list | NO | (5′->3′) | gene |
| 17 target | 2 | GTTTGAACTAACTGGACAC | 372 |
| regions on | 3 | TGATTTTACTCATCCATGT | 399 |
| Hif1α cDNA | 4 | CATGAGGAAATGAGAGAAA | 421 |
| | 5 | GAGAAATGCTTACACACAG | 434 |
| | 6 | CGAGGAAGAACTATGAACA | 532 |
| | 7 | GAACATAAAGTCTGCAACA | 546 |
| | 8 | TGATACCAACAGTAACCAA | 603 |
| | 9 | TCAGTGTGGGTATAAGAAA | 624 |
| | 10 | GCTGATTTGTGAACCCATT | 663 |
| | 11 | GCCGCTCAATTTATGAATA | 815 |
| | 12 | GCATTGTATGTGTGAATTA | 1001 |
| | 13 | TCAGGATCAGACACCTAGT | 1482 |
| | 14 | ATTTAGACTTGGAGATGTT | 1667 |
| | 15 | AGAGGTGGATATGTCTGGG | 931 |
| | 16 | CACCAAAGTGGAATCAGAA | 1125 |
| | 17 | TTCAAGTTGGAATTGGTAG | 1591 |
| | 18 | AAAGTCGGACAGCCTCACCAA | 1988 |

As used herein, the term ‘target mRNA’ refers to human Hif1α mRNA, Hif1αmRNA of the same line as human, and an alternative splice form thereof. Specifically, it may include Human: NM_{—}001530, NM_{—}181054 (splice form wherein bases of the positions from 2203 to 2248 are deleted in NM_{—}001530), Mus musculus: NM_{—}0010431, Macaca fascicularis: AB169332, and the like. Thus, the siRNA of the present invention may target Hif1α mRNA of human or the same line as human, or an alternative splice form thereof.

As used herein, the wording ‘targeting mRNA (or cDNA) region’ means that siRNA has a base sequence complementary to the base sequence of the whole or a part of the mRNA (or cDNA) region, for example, to 85˜100% of the whole base sequence, thus capable of specifically binding to the mRNA (or cDNA) region.

As used herein, the term ‘complementary’ or ‘complementarily’ means that both strands of polynucleotide may form a base pair. Both strands of complementary polynucleotide forms a Watson-Crick base pair to form double strands. When the base U is referred to herein, it may be substituted by the base T unless otherwise indicated.

Since the inhibition effect on Hif1α synthesis and/or expression and cancer therapeutic effect of the pharmaceutical composition of the present invention is achieved by effective inhibition on Hif1α synthesis and/or expression, siRNA contained in the pharmaceutical composition as an active ingredient may be double stranded siRNA of 15-30 bp that targets at least one of the specific mRNA regions as described above. The siRNA may have a symmetric structure having a blunt end without overhang, or it may have an asymmetric structure having an overhang of 1-5 nucleotides (nt) at 3′ end, 5′ end, or both ends. The nucleotide of the overhang may be any sequence, for example, 2 to 4 dTs (deoxythymidine), such as 2 dTs may be attached thereto.

According to preferable embodiment, the siRNA may include at least one selected from the group consisting of SEQ ID NOs. 19 to 22, 25 to 44, and 53 to 115. More specifically, the siRNA may be at least one selected from the group consisting of siRNA 1, siRNA 2, siRNA 4 to siRNA 13, and siRNA 18 to siRNA 50, as described in the following Table 2.

TABLE 2

| | SEQ | | | | |
|---|---|---|---|---|---|
| | ID | | | siRNA | |
| | NO | sequence (5′->3′) | Strand | indication | Modification |
| 17 Double- | 19 | GUUUGAACUAACUGGACACdTdT | Sense | siRNA 1 | |
| stranded | 20 | GUGUCCAGUUAGUUCAAACdTdT | Antisense | | |
| symmetric | 21 | UGAUUUUACUCAUCCAUGUdTdT | Sense | siRNA 2 | |
| siRNAs | 22 | ACAUGGAUGAGUAAAAUCAdTdT | Antisense | | |
| | 23 | CAUGAGGAAAUGAGAGAAAdTdT | Sense | siRNA 3 | |
| | 24 | UUUCUCUCAUUUCCUCAUGdTdT | Antisense | | |
| | 25 | GAGAAAUGCUUACACACAGdTdT | Sense | siRNA 4 | |
| | 26 | CUGUGUGUAAGCAUUUCUCdTdT | Antisense | | |
| | 27 | CGAGGAAGAACUAUGAACAdTdT | Sense | siRNA 5 | |
| | 28 | UGUUCAUAGUUCUUCCUCGdTdT | Antisense | | |
| | 29 | GAACAUAAAGUCUGCAACAdTdT | Sense | siRNA 6 | |
| | 30 | UGUUGCAGACUUUAUGUUCdTdT | Antisense | | |
| | 31 | UGAUACCAACAGUAACCAAdTdT | Sense | siRNA 7 | |
| | 32 | UUGGUUACUGUUGGUAUCAdTdT | Antisense | | |
| | 33 | UCAGUGUGGGUAUAAGAAAdTdT | Sense | siRNA 8 | |
| | 34 | UUUCUUAUACCCACACUGAdTdT | Antisense | | |
| | 35 | GCUGAUUUGUGAACCCAUUdTdT | Sense | siRNA 9 | |
| | 36 | AAUGGGUUCACAAAUCAGCdTdT | Antisense | | |
| | 37 | GCCGCUCAAUUUAUGAAUAdTdT | Sense | siRNA 10 | |
| | 38 | UAUUCAUAAAUUGAGCGGCdTdT | Antisense | | |
| | 39 | GCAUUGUAUGUGUGAAUUAdTdT | Sense | siRNA 11 | |
| | 40 | UAAUUCACACAUACAAUGCdTdT | Antisense | | |
| | 41 | UCAGGAUCAGACACCUAGUdTdT | Sense | siRNA 12 | |
| | 42 | ACUAGGUGUCUGAUCCUGAdTdT | Antisense | | |
| | 43 | AUUUAGACUUGGAGAUGUUdTdT | Sense | siRNA 13 | |
| | 44 | AACAUCUCCAAGUCUAAAUdTdT | Antisense | | |
| | 45 | AGAGGUGGAUAUGUCUGGGdTdT | Sense | siRNA 14 | |
| | 46 | CCCAGACAUAUCCACCUCUdTdT | Antisense | | |
| | 47 | CACCAAAGUGGAAUCAGAAdTdT | Sense | siRNA 15 | |
| | 48 | UUCUGAUUCCACUUUGGUGdTdT | Antisense | | |
| | 49 | UUCAAGUUGGAAUUGGUAGdTdT | Sense | siRNA 16 | |
| | 50 | CUACCAAUUCCAACUUGAAdTdT | Antisense | | |
| | 51 | AAAGUCGGACAGCCUCACCAA | Sense | siRNA 17 | |
| | 52 | UUGGUGAGGCUGUCCGACUUU | Antisense | | |
| 3 Double- | 53 | GGAAGAACUAUGAACA | Sense | siRNA 18 | |
| stranded | 28 | UGUUCAUAGUUCUUCCUCGdTdT | Antisense | | |
| asymmetric | 54 | GAUUUGUGAACCCAUU | Sense | siRNA 19 | |
| siRNAs | 36 | AAUGGGUUCACAAAUCAGCdTdT | Antisense | | |
| | 55 | UUGUAUGUGUGAAUUA | Sense | siRNA 20 | |
| | 40 | UAAUUCACACAUACAAUGCdTdT | Antisense | | |
| 30 | 56 | CGAGGAAGAACUAUGAACAdT*dT | Sense | siRNA21 | siRNA5- |
| Chemically | 57 | UGUUCAUAGUUCUUCCUCGdT*dT | Antisense | | mod1 |
| modified | 58 | CGAGGAAGAACUAUGAACAdT*dT | Sense | siRNA22 | siRNA5- |
| siRNAs | 59 | UGUUCAUAGUUCUUCCUCGdT*dT | Antisense | | mod2 |
| | 60 | CGAGGAAGAACUAUGAACAdT*dT | Sense | siRNA23 | siRNA5- |
| | 61 | UGUUCAUAGUUCUUCCUCGdT*dT | Antisense | | mod3 |
| | 62 | CGAGGAAGAACUAUGAACAdT*dT | Sense | siRNA24 | siRNA5- |
| | 63 | UGUUCAUAGUUCUUCCUCGdT*dT | Antisense | | mod4 |
| | 64 | CGAGGAAGAACuAuGAACAdT*dT | Sense | siRNA25 | siRNA5- |
| | 65 | UGuuCAuAGUUCuuCCuCGdT*dT | Antisense | | mod5 |
| | 66 | CGAGGAAGAACUAUGAACAdT*dT | Sense | siRNA26 | siRNA5- |
| | 67 | UGUUCAUAGUUCUUCCUCGdT*dT | Antisense | | mod6 |
| | 68 | CGAGGAAGAACUAUGAACAdT*dT | Sense | siRNA27 | siRNA5- |
| | 69 | UGUUCAUAGUUCUUCCUCGdT*dT | Antisense | | mod7 |
| | 70 | cGAGGAAGAAcuAuGAAcAdT*dT | Sense | siRNA28 | siRNA5- |
| | 71 | uGuucAuAGUcuuccucGdT*dT | Antisense | | mod8 |
| | 72 | CGAGGAAGAACUAUGAACAdT*dT | Sense | siRNA29 | siRNA5- |
| | 73 | UGUUCAUAGUUCUUCCUCGdT*dT | Antisense | | mod9 |
| | 74 | CGAGGAAGAACUAUGAACAdT*dT | Sense | siRNA30 | siRNA5- |
| | 75 | UGUUCAUAGUUCUUCCUCGdT*dT | Antisense | | mod10 |
| | 76 | GCUGAUUUGUGAACCCAUUdT*dT | Sense | siRNA31 | siRNA9- |
| | 77 | AAUGGGUUCACAAAUCAGCdT*dT | Antisense | | mod1 |
| | 78 | GCUGAUUUGUGAACCCAUUdT*dT | Sense | siRNA32 | siRNA9- |
| | 79 | AAUGGGUUCACAAAUCAGCdT*dT | Antisense | | mod2 |
| | 80 | GCUGAUUUGUGAACCCAUUdT*dT | Sense | siRNA33 | siRNA- |
| | 81 | AAUGGGUUCACAAAUCAGCdT*dT | Antisense | | mod3 |
| | 82 | GCUGAUUUGUGAACCCAUUdT*dT | Sense | siRNA34 | siRNA9- |
| | 83 | AAUGGGUUCACAAAUCAGCdT*dT | Antisense | | mod4 |
| | 84 | GCuGAuuuGuGAACCCAuudT*dT | Sense | siRNA35 | siRNA9- |
| | 85 | AAuGGGuuCACAAAuCAGCdT*dT | Antisense | | mod5 |
| | 86 | GCUGAUUUGUGAACCCAUUdT*dT | Sense | siRNA36 | siRNA9- |
| | 87 | AAUGGGUUCACAAAUCAGCdT*dT | Antisense | | mod6 |
| | 88 | GCUGAUUUGUGAACCCAUUdT*dT | Sense | siRNA37 | siRNA9- |
| | 89 | AAUGGGUUCACAAAUCAGCdT*dT | Antisense | | mod7 |
| | 90 | GcuGAuuuGUGAAcccAuudT*dT | Sense | siRNA38 | siRNA9- |
| | 91 | AAuGGGuucACAAAucAGcdT*dT | Antisense | | mod8 |
| | 92 | GCUGAUUUGUGAACCCAUUdT*dT | Sense | siRNA39 | siRNA9- |
| | 93 | AAUGGGUUCACAAAUCAGCdT*dT | Antisense | | mod9 |
| | 94 | GCUGAUUUGUGAACCCAUUdT*dT | Sense | siRNA40 | siRNA9- |
| | 95 | AAUGGGUUCACAAAUCAGCdT*dT | Antisense | | mod10 |
| | 96 | GCAUUGUAUGUGUGAAUUAdT*dT | Sense | siRNA41 | siRNA 11- |
| | 97 | UAAUUCACACAUACAAUGCdT*dT | Antisense | | mod1 |
| | 98 | GCAUUGUAUGUGUGAAUUAdT*dT | Sense | siRNA42 | siRNA 11- |
| | 99 | UAAUUCACACAUACAAUGCdT*dT | Antisense | | mod2 |
| | 100 | GCAUUGUAUGUGUGAAUUAdT*dT | Sense | siRNA43 | siRNA 11- |
| | 101 | UAAUUCACACAUACAAUGCdT*dT | Antisense | | mod3 |
| | 102 | GCAUUGUAUGUGUGAAUUAdT*dT | Sense | siRNA44 | siRNA 11- |
| | 103 | UAAUUCACACAUACAAUGCdT*dT | Antisense | | mod4 |
| | 104 | GCAuuGuAuGuGuGAAuuAdT*dT | Sense | siRNA45 | siRNA 11- |
| | 105 | UAAuuCACACAuACAAuGCdT*dT | Antisense | | mod5 |
| | 106 | GCAUUGUAUGUGUGAAUUAdT*dT | Sense | siRNA46 | siRNA 11- |
| | 107 | UAAUUCACACAUACAAUGCdT*dT | Antisense | | mod6 |
| | 108 | GCAUUGUAUGUGUGAAUUAdT*dT | Sense | siRNA47 | siRNA 11- |
| | 109 | UAAUUCACACAUACAAUGCdT*dT | Antisense | | mod7 |
| | 110 | GcAuuGuAuGuGuGAAuuAdT*dT | Sense | siRNA48 | siRNA 11- |
| | 111 | uAAuucAcACAuAcAAuGcdT*dT | Antisense | | mod8 |
| | 112 | GCAUUGUAUGUGUGAAUUAdT*dT | Sense | siRNA49 | siRNA 11- |
| | 113 | UAAUUCACACAUACAAUGCdT*dT | Antisense | | mod9 |
| | 114 | GCAUUGUAUGUGUGAAUUAdT*dT | Sense | siRNA50 | siRNA 11- |
| | 115 | UAAUUCACACAUACAAUGCdT*dT | Antisense | | mod10 |

TABLE 3

| notation | Introduced chemical modification |
|---|---|
| * | Phosphodiester bond → phosphorothioate bond |
| Underline | 2′-OH → 2′-O—Me |
| Lowercase letter | 2′-OH → 2′-F |
| Bold letter | ENA(2′-O, 4′-C ethylene bridged nucleotide) |

TABLE 4

| Structure | |
|---|---|
| name | siRNA chemical modification |
| mod1 | 2′-OH group of ribose of 1st and 2nd nucleic acids of antisense strand are substituted |
| | with 2′-O—Me |
| mod2 | in addition to mod1 modification, 2′-OH groups of riboses of 1st and 2nd nucleic acids of |
| | sense strand are substituted with 2′-O—Me |
| mod3 | in addition to mod2 modification, 2′-OH groups of riboses of all U containing nucleic |
| | acids of sense strand are substituted with 2′-O—Me |
| mod4 | in addition to mod3 modification, 2′-OH groups of riboses of all U containing nucleic |
| | acids of antisense strand are substituted with 2′-O—Me |
| mod5 | in addition to mod1 modification, 2′-OH groups of riboses of all G containing nucleic |
| | acids of sense and antisense strands are substituted with 2′-O—Me, and 2′-OH groups of |
| | riboses of all U containing nucleic acids of sense and antisense strands are substituted |
| | with 2′-F |
| mod6 | in addition to mod1 modification, 5′ end of sense strand is substituted with ENA(2′-O, 4′- |
| | C ethylene bridged nucleotide) |
| mod7 | 2′-OH group of 2^{nd} nucleic acid of 5′ end of antisense strand is substituted with 2′-O—Me |
| mod8 | 2′-OH groups of all U or C containing nucleic acids of sense and antisense strands are |
| | substituted with 2′-F |
| mod9 | 2′-OH groups of all G containing nucleic acids of sense strand are substituted with 2′-O—Me, |
| | and 2′-OH groups of all nucleic acids containing U of GU sequence, or 1^{st} U of |
| | UUU or UU sequence of antisense strand are substituted with 2′-O—Me |
| mod10 | 2′-OH groups of even-numbered nucleic acids of sense strand are substituted with 2′-O—Me, |
| | and 2′-OH groups of odd-numbered nucleic acids of antisense strand are substituted |
| | with 2′-O—Me |

In the Table 4, the modifications from mod1 to mod7 do not modify 10^{th} and 11^{th} bases of an antisense strand, and dTdT (phosphodiester bond) at 3′ end of sense and antisense strands of all siRNAs in the modifications of mod 1 to mod 10 are substituted with a phosphorotioate bond (3′-dT*dT, *: Phosphorothioate bond).

Since the siRNA has high sequence specificity for a specific target region of Hif1α mRNA transcript, it can specifically complementarily bind to the transcript of a target gene, thereby increasing. RNA interference activity, thus having excellent activity of inhibiting Hif1α expression and/or synthesis in cells. And, the siRNA has minimal immune inducing activity.

As described above, the siRNA of the present invention may be siRNA targeting at least one region of mRNA selected from the group consisting of SEQ ID NOs. 2, 3, and 5 to 14 of the Hif1α cDNA region of SEQ ID NO. 1. Preferably, the siRNA may comprise at least one nucleotide sequence selected from the group consisting of SEQ ID NOs. 19 to 22, 25 to 44, and 53 to 115, and more preferably, at least one selected from the group consisting of 45 siRNAs of SEQ ID NOs. 19 to 22, 25 to 44, and 53 to 115. The siRNA includes ribonucleic acid sequence itself, and a recombinant vector (expression vector) expressing the same. The expression vector may be a viral vector selected from the group consisting of a plasmid or an adeno-associated virus, a retrovirus, a vaccinia virus, an oncolytic adenovirus, and the like.

The pharmaceutical composition of the present invention may comprise the siRNA as an active ingredient and a pharmaceutically acceptable carrier. The pharmaceutically acceptable carrier may include any commonly used carriers, and for example, it may be at least one selected from the group consisting of water, a saline solution, phosphate buffered saline, dextrin, glycerol, ethanol, and the like, but not limited thereto.

The siRNA may be administered to mammals, preferably human, monkey, or rodents (mouse, rate), and particularly, to any mammals, for example human, who has diseases or conditions related to Hif1α expression, or requires inhibition of Hif1α expression.

To obtain Hif1α inhibition effect while minimizing undesirable side effects such as an immune response, and the like, the concentration of the siRNA in the composition or a dosage of the siRNA may be 0.001 to 1000 nM, preferably 0.01 to 100 nM, more preferably 0.1 to 10 nM, but not limited thereto.

The siRNA or the pharmaceutical composition containing the same may treat at least one cancer selected from the group consisting of various solid cancers (such as lung cancer, liver cancer, colorectal cancer, pancreatic cancer, stomach cancer, breast cancer, ovarian cancer, renal cancer, thyroid cancer, esophageal cancer, prostate cancer, brain cancer, and the like), skin cancer, osteosarcoma, soft tissue sarcoma, glioma, lymphoma, and the like.

Hereinafter, the structure and the designing process of the siRNA, and a pharmaceutical composition containing the same will be described in detail.

The siRNA may have a role that does not induce or do decrease the expression of protein by degrading Hif1α mRNA by RNAi pathway.

According to one embodiment, siRNA refers to small inhibitory RNA duplexes that induce RNA interference (RNAi) pathway. Specifically, siRNA is RNA duplexes comprising a sense strand and an antisense strand complementary thereto, wherein both strands comprise 15-30 bp, specifically 15-25 bp, more specifically 15-22 bp. The siRNA may comprise a double stranded region and a region where a single strand forms a hairpin or a stem-loop structure, or it may be duplexes of two separated strands. The sense strand may have identical sequence to the nucleotide sequence of a target gene mRNA sequence. A duplex forms between the sense strand and the antisense strand complementary thereto by Watson-Crick base pairing. The antisense strand of siRNA is captured in RISC(RNA-Induced Silencing Complex), and the RISC identifies the target mRNA which is complementary to the antisense strand, and then, induces cleavage or translational inhibition of the target mRNA.

According to one embodiment, the double stranded siRNA may have an overhang of 1 to 5 nucleotides at 3′ end, 5′ end, or both ends. Alternatively, it may have a blunt end truncated at both ends. Specifically, it may be siRNA described in US20020086356, and U.S. Pat. No. 7,056,704, which are incorporated herein by reference.

According to one embodiment, the siRNA comprises a sense strand and an antisense strand, wherein the sense strand and the antisense strand form a duplex of 15-30 bp, and the duplex may have a symmetrical structure having a blunt end without an overhang, or an asymmetric structure having an overhang of at least one nucleotide, for example 1-5 nucleotides. The nucleotides of the overhang may be any sequence, but 2 to 4 dTs (deoxythymidine), for example, 2 dTs may be attached thereto.

The antisense strand is hybridized with the target region of mRNA of SEQ ID NO. 1, under a physiological condition. The description ‘hybridized under physiological condition’ means that the antisense strand of the siRNA is in vivo hybridized with a specific target region of mRNA. Specifically, the antisense strand may have 85% or more sequence complementarity to the target mRNA region, where the target mRNA region is preferably at least one base sequence selected from SEQ ID NOs. 2, 3, and 5 to 14 as shown in Table 1, and more specifically, the antisense strand may comprise a sequence completely complementary to consecutive 15 to 30 bp, preferably consecutive 15 to 25 bp, more preferably consecutive 15 to 22 bp, within the base sequence of SEQ ID NO. 1. Still more preferably, the antisense strand of the siRNA may comprise a sequence completely complementary to at least one base sequence selected from SEQ ID NOs. 2, 3, and 5 to 14, as shown in Table 1.

According to one embodiment, the siRNA may have an asymmetric double stranded structure, wherein one strand is shorter than the other strand. Specifically, in the case of siRNA (small interfering RNA) molecule of double strands consisting of an antisense strand of 19 to 21 nucleotides (nt) and a sense strand of 15 to 19 nt having complementary sequence to the antisense (provided that if the antisense strand is 19 nt, the sense strand is not 19 nt), the siRNA may be an asymmetric siRNA having a blunt end at 5′ end of the antisense and a 1-5 nucleotides overhang (for example, (dT)n, n=1-5, preferably integer of 2-4) at 3′ end of the antisense. Specifically, it may be siRNA disclosed in WO09/078685.

In the treatment using siRNA, it is required to select an optimum base sequence having highest activity in the base sequence of the targeted gene. Specifically, according to one embodiment, to increase relationship between pre-clinical trials and clinical trial, it is preferable to design Hif1α siRNA comprising a conserved sequence between species. And, according to one embodiment, it is preferable to design such that the antisense strand binding to RISC may have high binding ability to RISC. Thus, it may be designed such that there may be difference between thermodynamic stabilities between a sense strand and an antisense strand, thus increasing RISC binding ability of the antisense strand that is a guide strand, while the sense strand does not bind to RISC. Specifically, GC content of the sense strand may not exceed 60%; 3 or more adenine/guanine bases may exist in the 15^{th} to 19^{th} positions from 5′ end of the sense strand; and G/C bases may be abundant in the 1^{st} to 7^{th} positions from 5′ end of the sense strand.

And, since due to repeated base sequences, internal sequences of siRNA itself may bind to each other and lower the ability of complementary binding to mRNA, it may be preferable to design such that less than 4 repeated base sequences exist. And, in the case of a sense strand consisting of 19 bases, to bind to mRNA of a target gene to properly induce degradation of the transcript, 3^{rd}, 10^{th}, and 19^{th} bases from 5′ end of the sense strand may be adenine.

Further, according to one embodiment, siRNA has minimized non-specific binding and immune response-inducing activity. The inducing of an immune response of interferon, and the like by siRNA mostly occurs through TLR7 (Toll-like receptor-7) that exists at endosome of antigen-presenting immune cells, and binding of siRNA to TLR7 occurs in a sequence specific manner like in GU rich sequences, and thus, it may be best to comprise a sequence that is not recognized by TLR7. Specifically, it may not have an immune response-inducing sequence such as 5′-GUCCUUCAA-3′ and 5′-UGUGU-3′, and have 70% or less complementarity to genes other than Hif1α.

Examples of the Hif1α cDNA target sequence include the nucleotides of the sequences described in the above Table 1. Based on the target sequences of Table 1, siRNA sequence may be designed such that siRNA length may be longer or shorter than the length of the target sequence, or nucleotides complementary to the DNA sequences may be added or deleted.

According to one embodiment of the invention, siRNA may comprise a sense strand and an antisense strand, wherein the sense strand and the antisense strand form double strands of 15-30 bp without an overhang, or at least one end may have an overhang of 1-5 nucleotides, and the antisense strand may be hybridized to the mRNA region corresponding to any one of SEQ ID NOs 2, 3, and 5 to 14, preferably SEQ ID NO 6, 10, 12, under physiological condition. Namely, the antisense strand comprises a sequence complementary to any one of SEQ ID NOs 2, 3, and 5 to 14, preferably to SEQ ID NOs 6, 10, 12. Thus, the Hif1α siRNA and the pharmaceutical composition containing the same of the present invention do not induce a harmful interferon response and yet inhibit expression of Hif1α gene.

The present invention inhibits expression of Hif1α in cells by complementary binding to the mRNA region corresponding to at least one sequence selected from the group consisting of SEQ ID NO 6 (5′-CGAGGAAGAACTATGAACA-3′), SEQ ID NO (5′-GCTGATTTGTGAACCCATT-3′), and SEQ ID NO 12 (5′-GCATTGTATGTGTGAATTA-3′).

The Hif1α siRNA according to specific embodiments of the invention are as described in the above Table 2.

According to one embodiment, the Hif1α siRNA may be at least one selected from the group consisting of siRNA 5 comprising a sense sequence of SEQ ID NO 27 and an antisense sequence of SEQ ID NO 28, siRNA 9 comprising a sense sequence of SEQ ID NO 35 and an antisense sequence of SEQ ID NO 36, siRNA 11 comprising a sense sequence of SEQ ID NO 39 and an antisense sequence of SEQ ID NO 40, siRNA 18 comprising a sense sequence of SEQ ID NO 53 and an antisense sequence of SEQ ID NO 28, siRNA 19 comprising a sense sequence of SEQ ID NO 54 and an antisense sequence of SEQ ID NO 36, and siRNA 20 comprising a sense sequence of SEQ ID NO 55 and an antisense sequence of SEQ ID NO 40.

Knockdown (Hif1α expression inhibition) may be confirmed by measuring change in the mRNA or protein level by quantitative PCR (qPCR) amplification, bDNA (branched DNA) assay, Western blot, ELISA, and the like. According to one embodiment, a liposome complex is prepared to treat cancer cell lines, and then, ribonucleic acid-mediated interference of expression may be confirmed by bDNA assay in mRNA stage.

The siRNA sequence of the present invention has low immune response inducing activity while effectively inhibiting synthesis or expression of Hif1α.

According to one embodiment, immune toxicity may be confirmed by treating human peripheral blood mononuclear cells (PBMC) with an siRNA-DOTAP (N-[1-(2,3-Dioleoyloxy)propyl]-N,N,N-trimethylammonium methylsulfate) complex, and then, measuring whether released cytokines of INF-α and INF-γ, tumor necrosis factor-α (TNF-α), interleukin-12 (IL-12), and the like are increased or not in the culture medium.

The siRNA may have a naturally occurring (unmodified) ribonucleic acid unit structure, or it may be chemically modified, and for example, it may be synthesized such that the sugar or base structure of at least one ribonucleic acid, a bond between ribonucleic acids may have at least one chemical modification. Through the chemical modification of siRNA, desirable effects such as improved resistance to nuclease, increased intracellular uptake, increased cell targeting (target specificity), increased stability, or decreased off-target effect such as decreased interferon activity, immune response and sense effect, and the like may be obtained without influencing the original RNAi activity.

The chemical modification method of siRNA is not specifically limited, and one of ordinary skills in the art may synthesize and modify the siRNA as desired by a method known in the art (Andreas Henschel, Frank Buchholz 1 and Bianca Habermann (2004) DEQOR: a web based tool for the design and quality control of siRNAs. Nucleic Acids Research 32(Web Server Issue): W113-W120).

For example, a phosphodiester bond of siRNA sense or antisense strand may be substituted with boranophosphate or phosphorothioate to increase resistance to nucleic acid degradation. For example, it may be introduced at 3′ or 5′ end or both ends of siRNA sense or antisense strand, preferably only at RNA terminus, for example, 3′ end overhang (for example, (dT)n, n=an integer of 1-5, preferably of 2-4).

For another example, ENA (Ethylene bridge nucleic acid) or LNA (Locked nucleic acid) may be introduced at 5′ or 3′ end, or both ends of siRNA sense or antisense strand, and preferably, it may be introduced at 5′ end of siRNA sense strand. Thereby, siRNA stability may be increased, and an immune response and non-specific inhibition may be reduced, without influencing the RNAi activity.

For yet another example, a 2′-OH group of ribose ring may be substituted with —NH₂ (amino group), —C-allyl(allyl group), —F (fluoro group), or —O-Me (or CH₃, methyl group). For example, 2′-OH group of ribose of 1st and 2nd nucleic acids of antisense strand may be substituted with 2′-O-Me, 2′-OH groups of ribose of 2^{nd} nucleic acid of antisense strand may be substituted with 2′-O-Me, or 2′-OH of riboses of guanine (G) or uridine (U) containing nucleotides may be substituted with 2′-O-Me (methyl group) or 2′-F (fluoro group).

In addition to the above described chemical modifications, various chemical modifications may be made, and only one chemical modification may be made or a plurality of chemical modifications may be made in combination.

According to one embodiment, chemical modification may be one of the chemical modifications of Table 4, and in Table 4, mod1 to mod7 may not modify in the 10^{th} and 11^{th} bases of the antisense strand, and dTdT (phosphodiester bond) at 3′ end of all siRNA sense and antisense strands of mod 1 to mod 10 may be substituted with a phosphorotioate bond (3′-dT*dT, *: Phosphorothioate bond).

In the chemical modification, it is preferable that the activity of knockdown of gene expression may not be reduced while stabilizing the double stranded structure of the siRNA, and thus, minimum modification may be preferred.

And, a ligand such as cholesterol, biotin, or cell penetrating peptide may be attached at 5′- or 3′-end of siRNA.

The siRNA of the present invention may be manufactured by in vitro transcription or by cleaving long double stranded RNA with dicer or other nuclease having similar activities. Alternatively, as described above, siRNA may be expressed through plasmid or a viral expression vector, and the like.

A candidate siRNA sequence may be selected by experimentally confirming whether or not a specific siRNA sequence induces interferon in human peripheral blood mononuclear cells (PBMC) comprising dendritic cells, and then, selecting sequences which do not induce an immune response.

Hereinafter, a drug delivery system (DDS) for delivering the siRNA will be described.

A nucleic acid delivery system may be utilized to increase intracellular delivery efficiency of siRNA.

The nucleic acid delivery system for delivering nucleic acid material into cells may include a viral vector, a non-viral vector, liposome, cationic polymer micelle, emulsion, solid lipid nanoparticles, and the like. The non-viral vector may have high delivery efficiency and long retention time. The viral vector may include a retroviral vector, an adenoviral vector, a vaccinia virus vector, an adeno-associated viral vector, an oncolytic adenovirus vector, and the like. The nonviral vector may include plasmid. In addition, various forms such as liposome, cationic polymer micelle, emulsion, solid lipid nanoparticles, and the like may be used. The cationic polymer for delivering nucleic acid may include natural polymer such as chitosan, atelocollagen, cationic polypeptide, and the like and synthetic polymer such as poly(L-lysine), linear or branched polyethylene imine (PEI), cyclodextrin-based polycation, dendrimer, and the like.

The siRNA or complex of the siRNA and nucleic acid delivery system (pharmaceutical composition) of the present invention may be in vivo or ex vivo introduced into cells for cancer therapy. As shown by the following Examples, if the siRNA or complex of the siRNA and nucleic acid delivery system of the present invention is introduced into cells, it may selectively inhibit the expression of Hif1α to decrease the expression of target protein Hif1α involved in oncogenesis, and thus, cancer cells may be killed and cancer may be treated.

The siRNA or a pharmaceutical composition comprising the same of the present invention may be formulated for topical, oral or parenteral administration, and the like. Specifically, the administration route of siRNA may be topical such as ocular, intravaginal, or intraanus, and the like, parenteral such as intarpulmonary, intrabronchial, nasal cavity, integument, intraendothelial, intravenous, intraarterial, subcutaneous, intraabdominal, intramuscular, intracranial (intrathecal or intraventricular), and the like, or oral, and the like. For topical administration, the siRNA or the pharmaceutical composition comprising the same may be formulated in the form of a patch, ointment, lotion, cream, gel, drop, suppository, spray, solution, powder, and the like. For parenteral administration, intrathecal or intraventricular administration, the siRNA or pharmaceutical composition containing the same may comprise a sterilized aqueous solution containing appropriate additives such as buffer, diluents, penetration enhancer, other pharmaceutically acceptable carriers or excipient.

Further, the siRNA may be mixed with an injectable solution and administered by intratumoral injection in the form of an injection, or it may be mixed with a gel or transdermal adhesive composition and directly spread or adhered to an affected area to be r administered by transdermal route. The injectable solution is not specifically limited, but preferably, it may be an isotonic aqueous solution or suspension, and may be sterilized and/or contain additives (for example, antiseptic, stabilizer, wetting agent, emulsifying agent, solubilizing agent, a salt for controlling osmotic pressure, buffer and/or liposome preparation). The gel composition may contain a conventional gel preparation such as carboxymethyl cellulose, methyl cellulose, acrylic acid polymer, carbopol, and the like and a pharmaceutically acceptable carrier and/or a liposome preparation. And, in the transdermal adhesive composition, an active ingredient layer may include an adhesion layer, an adsorption layer for absorbing sebum and a therapeutic drug layer, and the therapeutic drug layer may contain a pharmaceutically acceptable carrier and/or a liposome preparation, but not limited thereto.

Further, the pharmaceutical composition for treating cancer of the present invention may further comprise known anticancer chemotherapeutics in addition to the siRNA for inhibiting expression of Hif1α, and thereby, combined effects may be anticipated. The anticancer chemotherapeutics that may be used for combined administration with the siRNA for inhibiting the expression of Hif1α of the present invention may include cisplatin, carboplatin, oxaliplatin, doxorubicin, daunorubicin, epirubicin, idarubicin, mitoxantrone, valubicin, curcumin, gefitinib, erlotinib, cetuximab, lapatinib, trastuzumab, sunitinib, sorafenib, bevacizumab, bortezomib, temsirolimus, everolimus, vorinostat, irinotecan, topotecan, vinblastine, vincristine, docetaxel, paclitaxel, and a combination thereof.

Further, in addition to or separately from the combination with chemotherapeutics, siRNA for inhibiting expression of various growth factors (VEGF, EGF, PDGF, and the like), growth factor receptor and downstream signal transduction protein, viral oncogene, anticancer and drug resistant gene may be combined with the Hif1α siRNA, thereby simultaneously blocking various cancer pathway to maximize anticancer effect.

According to another embodiment of the invention, provided is a method for inhibiting expression and/or synthesis of Hif1α, comprising contacting an effective amount of the Hif1α siRNA with Hif1α-expressing cells. The cell may include any cells expressing Hif1α, for example, cancer cell, and it may include cells in the body of animals, preferably mammals, for example, human, monkey, rodents (mouse, rat), and the like, and cells separated from the body. For example, the method for inhibiting expression and/or synthesis of Hif1α may comprise providing a Hif1α-expressing cell separated from the body of animals; and contacting the siRNA with the Hif1α-expressing cells separated from the body. The Hif1α-expressing cells may be obtained by artificially culturing Hif1α-expressing cells separated from the body.

According to yet another embodiment, provided is a method for inhibiting growth of cancer cells, comprising contacting an effective amount of the Hif1α siRNA for inhibiting synthesis and/or expression of Hif1α with cancer cells. The cancer cells may be cells existing in the body of animals, preferably mammals, for example, human, monkey, rodents (mouse, rat), and the like, or cells separated from the body. For example, the method for inhibiting growth of cancer cells may comprise providing Hif1α-expressing cancer cells separated from the body of an animal; and contacting the siRNA with the Hif1α-expressing cancer cells separated from the body.

According to yet another embodiment, provided is a method of preventing and/or treating cancer, comprising administering an effective amount of the Hif1αsiRNA and/or the expression vector containing the siRNA to a patient in need of prevention and/or treatment of cancer. The method of preventing and/or treating cancer may further comprise identifying a patient in need of prevention and/or treatment of cancer before the administration.

The cancer that may be treated according to the present invention may be at least one selected from the group consisting of most of the solid cancer (lung cancer, liver cancer, colorectal cancer, pancreatic cancer, stomach cancer, breast cancer, ovarian cancer, renal cancer, thyroid cancer, esophageal cancer, prostate cancer, brain cancer), skin cancer, osteosarcoma, soft tissue sarcoma, glioma, lymphoma, and the like.

The patient may include mammals, preferably, human, monkey, rodents (mouse, rate, and the like), and the like, and particularly, it may include any mammals, for example, human having a disease or condition (for example, cancer) related to Hif1αexpression or requiring inhibition of Hif1α expression.

The effective amount of the siRNA according to the present invention refers to the amount required for administration in order to obtain the effect of inhibiting Hif1αexpression or synthesis or the resulting cancer cell growth inhibition and the effect of cancer therapy. Thus, it may be appropriately controlled depending on various factors including the kind or severity of disease, kind of administered siRNA, kind of dosage form, age, weight, general health state, gender and diet of a patient, administration time, administration route, and treatment period, combined drug such as combined chemotherapeutic reagents, and the like. For example, daily dose may be 0.001 mg/kg˜100 mg/kg, which may be administered at a time or divided several times.

The siRNA complementary to the base sequence of Hif1α transcript (mRNA) of the preset invention may inhibit the expression of Hif1α that is commonly expressed in cancer cells by RNA-mediated interference (RNAi) to kill the cancer cells, and thus, it may manifest excellent anticancer effect. And, it may minimize the induction of immune responses.

The RNAi technology using RNA-mediated interference, adopted in the present invention, is suggested as the most effective method of selectively inhibiting the expression of Hif1α with high potency and accurate gene selectivity. While the existing drugs inhibit the function of already expressed proteins, the RNAi technology which is a natural gene silencing pathway may selectively inhibit the expression of specific disease inducing proteins and degrade the mRNA which is a pre-stage of protein synthesis, and thus, cancer growth and metastasis may be inhibited without inducing side-effects, and it will become a more fundamental cancer therapy.

Further, by combining chemotherapy with siRNA to increase the sensitivity to chemotherapeutics, therapeutic activity may be maximized and side-effects may reduce, and by combining siRNA for inhibiting the expression of various growth factor (VEGF, EFG, PDGF, and the like), growth factor receptor and downstream signal transduction protein, viral oncogene, and anticancer agent resistant gene with the Hill a siRNA to simultaneously block various cancer pathways, anticancer effect may be maximized.

### EXAMPLE

Hereinafter, the present invention will be described referring to the following examples.

However, these examples are only to illustrate the invention, and the scope of the invention is not limited thereto.

### Example 1

### Design of Target Base Sequence to which siRNA for Inhibiting Hif1α Expression May Bind

Using siRNA design programs of siDesign Center (Dharmacon), BLOCK-iT™ RNAi Designer (Invitrogen), AsiDesigner (KRIBB), siDirect (University of Tokyo) and siRNA Target Finder (Ambion), a target base sequence to which siRNA may bind was derived from the Hif1α mRNA sequence (NM_{—}001530). In the following Table 5, sequences indicated as cDNA sequences are shown as target base sequences.

TABLE 5
Target base sequence
(cDNA sequence)

| SEQ | |
|---|---|
| ID | |
| NO | sequence (5′->3′) |
| 2 | GTTTGAACTAACTGGACAC |
| 3 | TGATTTTACTCATCCATGT |
| 4 | CATGAGGAAATGAGAGAAA |
| 5 | GAGAAATGCTTACACACAG |
| 6 | CGAGGAAGAACTATGAACA |
| 7 | GAACATAAAGTCTGCAACA |
| 8 | TGATACCAACAGTAACCAA |
| 9 | TCAGTGTGGGTATAAGAAA |
| 10 | GCTGATTTGTGAACCCATT |
| 11 | GCCGCTCAATTTATGAATA |
| 12 | GCATTGTATGTGTGAATTA |
| 13 | TCAGGATCAGACACCTAGT |
| 14 | ATTTAGACTTGGAGATGTT |
| 15 | AGAGGTGGATATGTCTGGG |
| 16 | CACCAAAGTGGAATCAGAA |
| 17 | TTCAAGTTGGAATTGGTAG |
| 18 | AAAGTCGGACAGCCTCACCAA |

### Example 2

### Manufacture of siRNA for Inhibiting Hif1α Expression

20 kinds of siRNA that may bind to the target base sequences designed in Example 1 were obtained from ST Pharm Co. Ltd (Korea). 20 kinds of siRNA are as described in Table 6, wherein 3′ end of both strands comprises dTdT.

TABLE 6
Base sequence of siRNA for inhibiting
Hif1α expression

| SEQ | | | |
|---|---|---|---|
| ID | | | siRNA |
| NO | sequence (5′->3′) | strand | indication |
| 19 | GUUUGAACUAACUGGACACdTdT | Sense | siRNA 1 |
| 20 | GUGUCCAGUUAGUUCAAACdTdT | Antisense | |
| 21 | UGAUUUUACUCAUCCAUGUdTdT | Sense | siRNA 2 |
| 22 | ACAUGGAUGAGUAAAAUCAdTdT | Antisense | |
| 23 | CAUGAGGAAAUGAGAGAAAdTdT | Sense | siRNA 3 |
| 24 | UUUCUCUCAUUUCCUCAUGdTdT | Antisense | |
| 25 | GAGAAAUGCUUACACACAGdTdT | Sense | siRNA 4 |
| 26 | CUGUGUGUAAGCAUUUCUCdTdT | Antisense | |
| 27 | CGAGGAAGAACUAUGAACAdTdT | Sense | siRNA 5 |
| 28 | UGUUCAUAGUUCUUCCUCGdTdT | Antisense | |
| 29 | GAACAUAAAGUCUGCAACAdTdT | Sense | siRNA 6 |
| 30 | UGUUGCAGACUUUAUGUUCdTdT | Antisense | |
| 31 | UGAUACCAACAGUAACCAAdTdT | Sense | siRNA 7 |
| 32 | UUGGUUACUGUUGGUAUCAdTdT | Antisense | |
| 33 | UCAGUGUGGGUAUAAGAAAdTdT | Sense | siRNA 8 |
| 34 | UUUCUUAUACCCACACUGAdTdT | Antisense | |
| 35 | GCUGAUUUGUGAACCCAUUdTdT | Sense | siRNA 9 |
| 36 | AAUGGGUUCACAAAUCAGCdTdT | Antisense | |
| 37 | GCCGCUCAAUUUAUGAAUAdTdT | Sense | siRNA 10 |
| 38 | UAUUCAUAAAUUGAGCGGCdTdT | Antisense | |
| 39 | GCAUUGUAUGUGUGAAUUAdTdT | Sense | siRNA 11 |
| 40 | UAAUUCACACAUACAAUGCdTdT | Antisense | |
| 41 | UCAGGAUCAGACACCUAGUdTdT | Sense | siRNA 12 |
| 42 | ACUAGGUGUCUGAUCCUGAdTdT | Antisense | |
| 43 | AUUUAGACUUGGAGAUGUUdTdT | Sense | siRNA 13 |
| 44 | AACAUCUCCAAGUCUAAAUdTdT | Antisense | |
| 45 | AGAGGUGGAUAUGUCUGGGdTdT | Sense | siRNA 14 |
| 46 | CCCAGACAUAUCCACCUCUdTdT | Antisense | |
| 47 | CACCAAAGUGGAAUCAGAAdTdT | Sense | siRNA 15 |
| 48 | UUCUGAUUCCACUUUGGUGdTdT | Antisense | |
| 49 | UUCAAGUUGGAAUUGGUAGdTdT | Sense | siRNA 16 |
| 50 | CUACCAAUUCCAACUUGAAdTdT | Antisense | |
| 51 | AAAGUCGGACAGCCUCACCAA | Sense | siRNA 17 |
| 52 | UUGGUGAGGCUGUCCGACUUU | Antisense | |
| 53 | GGAAGAACUAUGAACA | Sense | siRNA 18 |
| 28 | UGUUCAUAGUUCUUCCUCGdTdT | Antisense | |
| 54 | GAUUUGUGAACCCAUU | Sense | siRNA 19 |
| 36 | AAUGGGUUCACAAAUCAGCdTdT | Antisense | |
| 55 | UUGUAUGUGUGAAUUA | Sense | siRNA 20 |
| 40 | UAAUUCACACAUACAAUGCdTdT | Antisense | |

### Example 3

### Hif1α Expression Inhibition Test in Cancer Cell Line Using siRNA

Using each siRNA manufactured in Example 2, human lung cancer cell line (A549, ATCC) was transformed, and Hif1α expression was measured in the transformed cancer cell line.

### Example 3-1

### Culture of Cancer Cell Line

Human lung cancer cell line (A549) obtained from American Type Culture Collection (ATCC) was cultured at 37° C., and 5% (v/v) CO₂, using RPMI culture medium (GIBCO/Invitrogen, USA) containing 10% (v/v) fetal bovine serum, penicillin (100 units/ml) and streptomycin (100 ug/ml).

### Example 3-2

### Manufacture of a Complex of siRNA for Hif1α Expression Inhibition and Liposome

For 20 siRNAs designed and synthesized in Example 1, a complex of siRNA for Hif1α expression inhibition and liposome lipofectamine 2000 (Invitrogen) for delivering the same was prepared.

25 ul of Opti-MEM medium (Gibco) containing 10 nM siRNA and Opti-MEM medium containing 0.4 ul of lipofectamine 2000 (Invitrogen) per well were mixed in the same volume, and reacted at room temperature for 20 minutes to prepare a complex of siRNA, and liposome.

### Example 3-3

### Inhibition of Hif1α mRNA Expression in Cancer Cell Line Using Hif1α Targeting siRNA

The lung cancer cell line cultured in Example 3-1 was seeded in a 96 well-plate at 10⁴ cells per well. After 24 hours, the medium was removed, and Opti-MEM medium was added in an amount of 50 μl per well. 50 μl of the complex composition of siRNA and liposome prepared in Example 3-2 was added, and cultured in a cell incubator while maintaining at 37° C. and 5% (v/v) CO₂ for 24 hours.

To calculate IC₅₀ value, which is a drug concentration for 50% inhibition of Hif1α mRNA expression, A549 cell line was treated with each siRNA of the 7 concentrations between 0.001 nM to 10 nM.

### Example 3-4

### Quantitative Analysis of Hif1α mRNA_Lung Cancer Cell

The expression degree of Hif1α mRNA, of which expression was inhibited by the siRNA liposome complex, was measured by bDNA analysis using Quantigene 2.0 system (Panomics, Inc.).

After the cells were treated with the siRNA liposome complex for 24 hours, mRNA was quantified. According to manufacturer's protocol, 100 μl of a lysis mixture (Panomics, Quantigene 2.0 bDNA kit) was treated per well of 96-well plate to lysis the cells at 50° C. for 1 hour. Probe specifically binding to Hif1α mRNA ((Panomics, Cat.# SA-11598) was purchased from Panomics, Inc., and mixed together with 80 μl of the obtained cell sample in a 96 well plate. Reaction was performed at 55° C. for 16 to 20 hours so that mRNA could be immobilized in the well and bind to the probe. Subsequently, 100 μl of the amplification reagent of the kit was introduced in each well, reacted and washed, which process was performed in two stages. 100 μl of the third amplification reagent was introduced and reacted at 50° C., and then, 100 μl of a luminescence inducing reagent was introduced, and after 5 minutes, luciferin value was measured by luminescence detector (Bio-Tek, Synergy-HT) to calculate percent value compared to the luminescence value of control (100%) which was treated with lipofectamine only. The percent indicates Hif1α mRNA expression rates of the control and each siRNA-treated test groups.

In human lung cancer cell line A549, relative value of luciferin value of test group treated with 10 nM Hif1α siRNA liposome complex was calculated compared to luciferin value of control treated with liposome only, to measure the level of Hif1αmRNA expression in A549 cell line transformed with siRNA, and the results are described in the following Table 7.

TABLE 7
Relative expression rate of Hif1α mRNA
in human lung cancer cell line (A549)
treated with 10 nM siRNA

| SEQ | | | Hif1α mRNA |
|---|---|---|---|
| ID | | siRNA | expression |
| NO | sequence (5′->3′) | No. | rate (%) |
| 2 | GTTTGAACTAACTGGACAC | 1 | 50.3 |
| 3 | TGATTTTACTCATCCATGT | 2 | 56.0 |
| 4 | CATGAGGAAATGAGAGAAA | 3 | 80.5 |
| 5 | GAGAAATGCTTACACACAG | 4 | 46.2 |
| 6 | CGAGGAAGAACTATGAACA | 5 | 29.6 |
| 7 | GAACATAAAGTCTGCAACA | 6 | 45.1 |
| 8 | TGATACCAACAGTAACCAA | 7 | 46.4 |
| 9 | TCAGTGTGGGTATAAGAAA | 8 | 53.8 |
| 10 | GCTGATTTGTGAACCCATT | 9 | 26.1 |
| 11 | GCCGCTCAATTTATGAATA | 10 | 49.9 |
| 12 | GCATTGTATGTGTGAATTA | 11 | 27.8 |
| 13 | TCAGGATCAGACACCTAGT | 12 | 46.9 |
| 14 | ATTTAGACTTGGAGATGTT | 13 | 56.3 |
| 15 | AGAGGTGGATATGTCTGGG | 14 | 81.7 |
| 16 | CACCAAAGTGGAATCAGAA | 15 | 73.7 |
| 17 | TTCAAGTTGGAATTGGTAG | 16 | 66.7 |
| 18 | AAAGTCGGACAGCCTCACCAA | 17 | 57.4 |

In Table 7, SEQ ID NOs. 2, 3, and 5 to 14 (siRNA NOs. 1, 2 and 4 to 13) correspond to Examples of the present invention, and SEQ ID NOs. 4 and 15 to 18 (siRNA Nos. 3 and 14 to 17) are presented as Comparative Examples. As shown in Table 7, as a result of examining the expression of Hif1α mRNA in the cell line transfected with total 17 kinds of siRNA, 12 kinds of siRNA of the present invention exhibited excellent inhibition effect compared to 5 kinds of siRNA of Comparative Examples. Specifically, among the 12 kinds of siRNA of the present invention, 9 kinds of siRNA exhibited more than 40% and less than 70% of inhibition rate (expression rate of more than 30% and less than 60%), and 3 kinds of siRNA exhibited 70% or more inhibition rate (expression rate of less than 30%).

For the 3 kinds of siRNA 5, 9 and 11 having excellent gene expression inhibition effect in Table 7, the effect of decreasing Hif1α mRNA expression was examined in the range of 10 nM of 0.001 nM using A549 cell line to calculate IC₅₀, and the results are described in the following Table 8. The IC₅₀ value was calculated using KC4 software supported by SofrMax pro software Biotek (Synergy-HT ELISA equipment) model supported by Spectra Max 190 (ELISA equipment) model. The IC₅₀ values of siRNA 5, 9 and 11 are shown about 4 to 500 time lower than those of siRNA 3 and 16.

TABLE 8
IC₅₀(nM) in A549 cell line

| | | corresponding | |
|---|---|---|---|
| siRNA | | mRNA | A549 |
| SEQ ID NO | siRNA No. | SEQ ID NO | (IC₅₀: nM) |
| 27, 28 | 5 | 6 | 0.02 |
| 35, 36 | 9 | 10 | 0.04 |
| 39, 40 | 11 | 12 | 0.02 |
| 23, 24 | 3 | 4 | >10 |
| 49, 50 | 16 | 17 | 0.16 |

### Example 3-5

### Hif1α mRNA Inhibition Effect of Asymmetric siRNA_Lung Cancer Cell

Lung cancer cell line A549 was respectively treated with each 10 nM of siRNA 5, 9 and 11 of a symmetric structure and siRNA 18, 19 and 20 of an asymmetric structure with sense strand shorter than antisense strand, which target SEQ ID NO. 6, 10, or 12, and Hif1α mRNA inhibition effect was examined, and the results are described in the following Table 9. The experimental method was the same as Examples 3-4.

TABLE 9
Hif1α mRNA expression rate according to structure modification

| siRNA | | Structural | |
|---|---|---|---|
| SEQ ID NO | siRNA No. | feature | Hif1α mRNA % |
| 27, 28 | 5 | Symmetric | 12.4 |
| 53, 28 | 18 | Asymmetric | 18.8 |
| 35, 36 | 9 | Symmetric | 9.2 |
| 54, 36 | 19 | Asymmetric | 6.2 |
| 39, 40 | 11 | Symmetric | 27.4 |
| 55, 42 | 20 | Asymmetric | 30.1 |

As shown in the Table 9, if SEQ ID NOs. 6, 10, and 12 are targeted, in asymmetric siRNA, Hif1α expression may be also effectively inhibited to a similar degree to symmetric siRNA.

### Example 4

### Chemical Modification of siRNA

Chemically modified siRNA 5, 9, and 11 were manufactured.

As shown in the following Table 10, 10 kinds of chemically modified siRNA were designed, wherein the chemical modification was made using 2′-O-Me, phosphorothioate bond, 2′-F, or by introducing ENA (Ethylene bridge nucleic acid) at the end. The chemically modified siRNA was synthesized by ST Pharm Co. Ltd (Korea).

TABLE 10
Chemically modified siRNA

| | SEQ | | | | |
|---|---|---|---|---|---|
| | ID | | | siRNA | |
| | NO | sequence (5′->3′) | strand | indication | Modification |
| Chemically | 56 | CGAGGAAGAACUAUGAACAdT*dT | Sense | siRNA21 | siRNA5- |
| modified | 57 | UGUUCAUAGUUCUUCCUCGdT*dT | Antisense | | mod1 |
| siRNA | 58 | CGAGGAAGAACUAUGAACAdT*dT | Sense | siRNA22 | siRNA5- |
| (30) | 59 | UGUUCAUAGUUCUUCCUCGdT*dT | Antisense | | mod2 |
| | 60 | CGAGGAAGAACUAUGAACAdT*dT | Sense | siRNA23 | siRNA5- |
| | 61 | UGUUCAUAGUUCUUCCUCGdT*dT | Antisense | | mod3 |
| | 62 | CGAGGAAGAACUAUGAACAdT*dT | Sense | siRNA24 | siRNA5- |
| | 63 | UGUUCAUAGUUCUUCCUCGdT*dT | Antisense | | mod4 |
| | 64 | CGAGGAAGAACuAuGAACAdT*dT | Sense | siRNA25 | siRNA5- |
| | 65 | UGuuCAuAGUUCuuCCuCGdT*dT | Antisense | | mod5 |
| | 66 | CGAGGAAGAACUAUGAACAdT*dT | Sense | siRNA26 | siRNA5- |
| | 67 | UGUUCAUAGUUCUUCCUCGdT*dT | Antisense | | mod6 |
| | 68 | CGAGGAAGAACUAUGAACAdT*dT | Sense | siRNA27 | siRNA5- |
| | 69 | UGUUCAUAGUUCUUCCUCGdT*dT | Antisense | | mod7 |
| | 70 | cGAGGAAGAAcuAuGAAcAdT*dT | Sense | siRNA28 | siRNA5- |
| | 71 | uGuucAuAGUcuuccucGdT*dT | Antisense | | mod8 |
| | 72 | CGAGGAAGAACUAUGAACAdT*dT | Sense | siRNA29 | siRNA5- |
| | 73 | UGUUCAUAGUUCUUCCUCGdT*dT | Antisense | | mod9 |
| | 74 | CGAGGAAGAACUAUGAACAdT*dT | Sense | siRNA30 | siRNA5- |
| | 75 | UGUUCAUAGUUCUUCCUCGdT*dT | Antisense | | mod10 |
| | 76 | GCUGAUUUGUGAACCCAUUdT*dT | Sense | siRNA31 | siRNA9- |
| | 77 | AAUGGGUUCACAAAUCAGCdT*dT | Antisense | | mod1 |
| | 78 | GCUGAUUUGUGAACCCAUUdT*dT | Sense | siRNA32 | siRNA9- |
| | 79 | AAUGGGUUCACAAAUCAGCdT*dT | Antisense | | mod2 |
| | 80 | GCUGAUUUGUGAACCCAUUdT*dT | Sense | siRNA33 | siRNA9- |
| | 81 | AAUGGGUUCACAAAUCAGCdT*dT | Antisense | | mod3 |
| | 82 | GCUGAUUUGUGAACCCAUUdT*dT | Sense | siRNA34 | siRNA9- |
| | 83 | AAUGGGUUCACAAAUCAGCdT*dT | Antisense | | mod4 |
| | 84 | GCuGAuuuGuGAACCCAuudT*dT | Sense | siRNA35 | siRNA9- |
| | 85 | AAuGGGuuCACAAAuCAGCdT*dT | Antisense | | mod5 |
| | 86 | GCUGAUUUGUGAACCCAUUdT*dT | Sense | siRNA36 | siRNA9- |
| | 87 | AAUGGGUUCACAAAUCAGCdT*dT | Antisense | | mod6 |
| | 88 | GCUGAUUUGUGAACCCAUUdT*dT | Sense | siRNA37 | siRNA9- |
| | 89 | AAUGGGUUCACAAAUCAGCdT*dT | Antisense | | mod7 |
| | 90 | GcuGAuuuGUGAAcccAuudT*dT | Sense | siRNA38 | siRNA9- |
| | 91 | AAuGGGuucACAAAucAGcdT*dT | Antisense | | mod8 |
| | 92 | GCUGAUUUGUGAACCCAUUdT*dT | Sense | siRNA39 | siRNA9- |
| | 93 | AAUGGGUUCACAAAUCAGCdT*dT | Antisense | | mod9 |
| | 94 | GCUGAUUUGUGAACCCAUUdT*dT | Sense | siRNA40 | siRNA9- |
| | 95 | AAUGGGUUCACAAAUCAGCdT*dT | Antisense | | mod10 |
| | 96 | GCAUUGUAUGUGUGAAUUAdT*dT | Sense | siRNA41 | siRNA 11- |
| | 97 | UAAUUCACACAUACAAUGCdT*dT | Antisense | | mod1 |
| | 98 | GCAUUGUAUGUGUGAAUUAdT*dT | Sense | siRNA42 | siRNA 11- |
| | 99 | UAAUUCACACAUACAAUGCdT*dT | Antisense | | mod2 |
| | 100 | GCAUUGUAUGUGUGAAUUAdT*dT | Sense | siRNA43 | siRNA 11- |
| | 101 | UAAUUCACACAUACAAUGCdT*dT | Antisense | | mod3 |
| | 102 | GCAUUGUAUGUGUGAAUUAdT*dT | Sense | siRNA44 | siRNA 11- |
| | 103 | UAAUUCACACAUACAAUGCdT*dT | Antisense | | mod4 |
| | 104 | GCAuuGuAuGuGuGAAuuAdT*dT | Sense | siRNA45 | siRNA 11- |
| | 105 | UAAuuCACACAuACAAuGCdT*dT | Antisense | | mod5 |
| | 106 | GCAUUGUAUGUGUGAAUUAdT*dT | Sense | siRNA46 | siRNA 11- |
| | 107 | UAAUUCACACAUACAAUGCdT*dT | Antisense | | mod6 |
| | 108 | GCAUUGUAUGUGUGAAUUAdT*dT | Sense | siRNA47 | siRNA 11- |
| | 109 | UAAUUCACACAUACAAUGCdT*dT | Antisense | | mod7 |
| | 110 | GcAuuGuAuGuGuGAAuuAdT*dT | Sense | siRNA48 | siRNA 11- |
| | 111 | uAAuucAcACAuAcAAuGcdT*dT | Antisense | | mod8 |
| | 112 | GCAUUGUAUGUGUGAAUUAdT*dT | Sense | siRNA49 | siRNA 11- |
| | 113 | UAAUUCACACAUACAAUGCdT*dT | Antisense | | mod9 |
| | 114 | GCAUUGUAUGUGUGAAUUAdT*dT | Sense | siRNA50 | siRNA 11- |
| | 115 | UAAUUCACACAUACAAUGCdT*dT | Antisense | | mod10 |

TABLE 11
notation of chemical modification

| notation | Introduced chemical modification |
|---|---|
| * | Phosphodiester bond → phosphorothioate bond |
| underline | 2′-OH → 2′-O—Me |
| Lower case letter | 2′-OH → 2′-F |
| Bold letter | ENA(2′-O, 4′-C ethylene bridged nucleotide) |

TABLE 12
Chemical modification of siRNA

| modification | Chemical modification of siRNA |
|---|---|
| mod1 | 2′-OH group of ribose of 1st and 2nd nucleic acids of antisense strand are |
| | substituted with 2′-O—Me |
| mod2 | in addition to mod1 modification, 2′-OH groups of riboses of 1st and 2nd nucleic |
| | acids of sense strand are substituted with 2′-O—Me |
| mod3 | in addition to mod2 modification, 2′-OH groups of riboses of all U containing |
| | nucleic acids of sense strand are substituted with 2′-O—Me |
| mod4 | in addition to mod3 modification, 2′-OH groups of riboses of all U containing |
| | nucleic acids of antisense strand are substituted with 2′-O—Me) |
| mod5 | in addition to mod1 modification, 2′-OH groups of riboses of all G containing |
| | nucleic acids of sense and antisense strands are substituted with 2′-O—Me, and 2′- |
| | OH groups of riboses of all U containing nucleic acids of sense and antisense |
| | strands are substituted with 2′-F |
| mod6 | in addition to mod1 modification, 5′ end of sense strand is substituted with ENA(2′- |
| | O, 4′-C ethylene bridged nucleotide) |
| mod7 | 2′-OH group of 2^{nd} nucleic acid of 5′ end of antisense strand is substituted with 2′- |
| | O—Me |
| mod8 | 2′-OH groups of all U or C containing nucleic acids of sense and antisense strands |
| | are substituted with 2′-F |
| mod9 | 2′-OH groups of all G containing nucleic acids of sense strand are substituted with |
| | 2′-O—Me, and 2′-OH groups of all nucleic acids containing U of GU sequence, or 1^{st} |
| | U of UUU or UU sequence of antisense strand are substituted with 2′-O—Me |
| mod10 | 2′-OH groups of even-numbered nucleic acids of sense strand are substituted with |
| | 2′-O—Me, and 2′-OH groups of odd-numbered nucleic acids of antisense strand are |
| | substituted with 2′-O—Me |

Wherein mod1 to mod7, do not modify 10^{th} and 11^{th} bases of antisense strand, and dTdT (phosphodiester bond) at 3′ end of all siRNA sense and antisense strands of mod 1 to mod 10 is substituted with a phosphorotioate bond (3′-dT*dT, *: Phosphorothioate bond).

### Example 5

### mRNA Inhibition Effect of Chemically Modified siRNA in Cancer Cell Line

To confirm whether or not the chemically modified siRNA of Example 4 maintains mRNA inhibiting activity in cancer cell line, unmodified siRNA (siRNA 5, 9 and 11) and 30 chemically modified siRNA of siRNA 21 to 50 were respectively formulated into a liposome complex as Example 3-2, and transfected to human lung cancer cell line (A549, ATCC) (10 nM siRNA), the Hif1α expression in the transfected cancer cell line was quantitatively analyzed in the same manner as Example 3-4, and the results are described in the following Table 13.

TABLE 13
Hif1α mRNA expression rate (%) in A549 cell line treated with 10 nM of
chemically modified siRNA

| | siRNA No. 5 | siRNA No. 9 | siRNA No. 11 |
|---|---|---|---|
| mod0 | 14.9 | 8.1 | 8.9 |
| mod1 | 46.3 | 8.6 | 17.6 |
| mod2 | 37.2 | 7.9 | 16.2 |
| mod3 | 23.0 | 61.3 | 10.9 |
| mod4 | 16.3 | 67.1 | 35.9 |
| mod5 | 6.2 | 20.2 | 8.0 |
| mod6 | 5.6 | 6.5 | 12.9 |
| mod7 | 4.1 | 7.0 | 11.1 |
| mod8 | 4.0 | 7.8 | 10.1 |
| mod9 | 6.0 | 6.7 | 8.9 |
| mod10 | 7.7 | 9.6 | 8.8 |

(Original siRNA that is not chemically modified is indicated as mod0.)

As shown in the Table 13, even when siRNA 5, 9 and 11 were chemically modified, the mRNA inhibition effects were maintained in cancer cell line. Particularly, mod5, mod6, mod7, mod8, mod9, and mod10 exhibited effects equivalent to or better than the effect of unmodified siRNA.

### Example 6

### Effect on Immunnoactive Cytokine Release

To evaluate whether or not the siRNA of the present invention has immune toxicity, experiment was conducted by the following process.

### Example 6-1

### Preparation of Peripheral Blood Mononuclear Cell

Human peripheral blood mononuclear cell (PBMC) was separated from blood supplied from healthy volunteer at experiment day using Histopaque 1077 reagent (Sigma, St Louis, Mo., USA) by density gradient centrifugation (Boyum A. Separation of leukocytes from blood and bone marrow. Scand J Clin Lab Invest 21(Supp197):77, 1968). The blood was carefully introduced on the Histopaque 1077 reagent seeded in a 15 ml tube at 1:1 ratio (by weight) so as not to be mixed with each other. After centrifugation at room temperature, 400×g, only a PBMC containing layer was separated with a sterilized pipet. Into the tube containing the separated PBMC, 10 ml of phosphate buffered saline (PBS) was transferred, and then, the mixture was centrifuged at 250×g for 10 minutes, and PBMC was additionally washed twice with 5 ml of PBS. The separated PBMC was suspended with serum-free x-vivo 15 medium (Lonza, Walkersville, Md., USA) to a concentration of 4×10⁶ cells/ml, and seeded in an amount of 100 ul per well in a 96-well plate.

### Example 6-2

### Formulation of siRNA-DOTAP Complex

A complex of siRNA-DOTAP for transfection of PBMC cells prepared in Example 6-1 was prepared as follows. 5 ul of a DOTAP transfection reagent (ROCHE, Germany) and 45 ul of x-vivo 15 medium, and 1 ul (50 uM) of mod1 to mod10 chemically modified siRNA 5, 9 11 and 49 ul of x-vivo 15 medium were respectively mixed, and then, reacted at room temperature for 10 minutes. After 10 minutes, the DOTAP containing solution and the siRNA containing solution were mixed and reacted at a temperature of 20 to 25° C. for 20 minutes to prepare a siRNA-DOTAP complex.

### Example 6-3

### Cell Culture

To 100 ul of the seeded PBMC culture solution of Example 6-1, the siRNA-DOTAP complex prepared according to Example 6-2 was added in an amount of 100 ul per well (siRNA final concentration 250 nM), and then, cultured in a CO₂ incubator of 37° C. for 18 hours. As control, cell culture groups not treated with the siRNA-DOTAP complex and cell culture groups treated with DOTAP only without siRNA were used. And, materials known to induce an immune response instead of siRNA, i.e., Poly I:C (Polyinosinic-polycytidylic acid potassium salt, Sigma, USA) and siApoB-1 siRNA (sense GUC AUC ACA CUG AAU ACC AAU (SEQ ID NO 116), antisense: *AUU GGU AUU CAG UGU GAU GAC AC, *: 5′ phosphates (SEQ ID NO 117), ST Pharm Co. Ltd.) were formulated into a complex with DOTAP by the same method as Example 6-2, and cell culture groups were treated therewith and used as positive control. After culture, only cell supernatant was separated.

### Example 6-4

### Measurement of Immune Activity

To measure the immune toxicity, peripheral blood mononuclear cells were treated with the siRNA-DOTAP complex as Example 6-3 and released cytokine was quantified. The contents of interferon alpha (INF-α) and interferon gamma (INF-γ), tumor necrosis factor (TNF-α), and interleukin-12 (IL-12) released in the supernatant were measured using Procarta Cytokine assay kit (Affymetrix, USA). Specifically, 50 ul of bead to which antibody to cytokine was attached (antibody bead) was moved to a filter plate and washed with wash buffer once, and then, 50 ul of supernatant of the PMBC culture solution and a cytokine standard solution were added and incubated at room temperature for 60 minutes while shaking at 500 rpm. The measuring device and samples including the bead to which antibody to cytokine was attached, wash buffer, and cytokine standard solution, which were included in Procarta Cytokine assay kit, were used.

Then, the solution was washed with washing buffer once, 25 ul of detection antibody included in the kit was added, and incubated at room temperature for 30 minutes while shaking at 500 rpm. Again, the reaction solution was removed under reduced pressure and washed, and then, 50 ul of streptavidin-PE (streptavidin phycoerythrin) included in the kit was added, and incubated at room temperature for 30 minutes while shaking at 500 rpm, and then, the reaction solution was removed and washed three times. 120 ul of reading buffer was added and the reaction solution was shaken at 500 rpm for 5 minutes, and then, PE fluorescence per cytokine bead was measured using Luminex equipment ((Bioplex luminex system, Biorad, USA). The cytokine concentration (pg/ml) released in the cell culture media when PBMC was treated with each 250 nM of siRNA is described in the following Table 14. The cytokine concentration in the sample was calculated from a standard calibration curve of 1:2˜220,000 pg/ml range.

TABLE 14
Cytokine concentration released in cell culture media when PBMC
is treated with 250 nM of chemically modified siRNA (pg/ml)

| | Test group | INF-alpha | INF-gamma | IL-12 | TNF-alpha |
|---|---|---|---|---|---|
| Control | MEDIUM | 2.56 | <2.44 | 4.82 | 10.9 |
| | DOTAP | 50.42 | <2.44 | 24.04 | 50.59 |
| | siApoB-1 | 713.03 | 3.06 | 51.36 | 77.4 |
| | Poly I:C | 255.95 | 38.86 | 2435.26 | 8629.78 |
| siRNA 5 | mod 1 | 122.31 | <2.44 | 33.14 | 46.05 |
| | mod 2 | 167.79 | <2.44 | 22.96 | 42.66 |
| | mod 3 | 45.29 | <2.44 | 42.18 | 30.33 |
| | mod 4 | 77.75 | <2.44 | 41.39 | 36.81 |
| | mod 5 | 54.52 | <2.44 | 39.8 | 38.17 |
| | mod 6 | 168.97 | <2.44 | 41.39 | 42.66 |
| | mod 7 | 121 | <2.44 | 46.04 | 46.49 |
| | mod 8 | 27.4 | <2.44 | 42.18 | 31.9 |
| | mod 9 | 47.65 | <2.44 | 40.6 | 31.43 |
| | mod 10 | 77.75 | <2.44 | 35.69 | 33.75 |
| siRNA 9 | mod 1 | 119.69 | <2.44 | 31.39 | 37.87 |
| | mod 2 | 56.75 | <2.44 | 33.14 | 46.05 |
| | mod 3 | 56.19 | <2.44 | 34.85 | 40.88 |
| | mod 4 | 64.34 | <2.44 | 37.36 | 39.83 |
| | mod 5 | 55.64 | <2.44 | 31.39 | 35.9 |
| | mod 6 | 87.59 | <2.44 | 61.75 | 63.18 |
| | mod 7 | 117.93 | <2.44 | 27.77 | 45.47 |
| | mod 8 | 65.4 | <2.44 | 40.6 | 48.97 |
| | mod 9 | 57.85 | <2.44 | 44.51 | 48.97 |
| | mod 10 | 48.82 | <2.44 | 27.77 | 47.81 |
| siRNA 11 | mod 1 | 513.7 | <2.44 | 25.89 | 42.81 |
| | mod 2 | 187.98 | <2.44 | 51.97 | 48.1 |
| | mod 3 | 107.21 | <2.44 | 47.55 | 35.59 |
| | mod 4 | 46.48 | <2.44 | 61.75 | 36.81 |
| | mod 5 | 52.26 | <2.44 | 83.96 | 44.59 |
| | mod 6 | 456.65 | <2.44 | 36.53 | 56.43 |
| | mod 7 | 454.57 | <2.44 | 21.95 | 48.68 |
| | mod 8 | 81.24 | <2.44 | 30.5 | 37.87 |
| | mod 9 | 79.75 | <2.44 | 50.51 | 47.08 |
| | mod 10 | 37.96 | <2.44 | 34.85 | 36.51 |

In Table 14, ‘Medium’ represents non-treated control, ‘DOTAP’ represents only DOTAP-treated group, ‘POLY I:C’ or ‘siApoB-1’ represents positive control group, ‘siRNA 5’ represents test group wherein the siRNA of SEQ ID NOs 27 and 28 are chemically modified as indicated, ‘siRNA 9’ represents test group wherein the siRNA of SEQ ID NOs. 35 and 36 are chemically modified as indicated, and ‘siRNA 11’ represents test group wherein the siRNA of SEQ ID NOs. 39 and 40 are chemically modified as indicated.

The chemically modified mod 1˜10 exhibited small increase in interferon alpha value, and little change or very small increase in the other cytokines. The value of interferon alpha remarkably decreases in the order of mod1→mod 2→mod 3, mod 4, mod 5, mod 8, mod 9, mod 10 to a level of only DOTAP-treated group, and thus, the chemically modified siRNA 5, 9 and 11 of the present invention may decrease immune activity.

### Example 7

### Inhibition of Off-Target Effect by Sense Strand of Chemically Modified siRNA

The following experiment was conducted to examine whether or not off-target effect by sense strand may be removed through chemical modification of siRNA.

The degree of off-target effect by sense strand can be seen by confirming that if a sense strand binds to RISC and acts on a sequence having a base sequence complementary to the sense strand, the amount of luciferase expressed by firefly Luciferase plasmid having a sequence complementary to the sense strand decreases compared to the cell that is not treated with siRNA. And, for cells treated with firefly luciferase plasmid having a sequence complementary to antisense, the degree of maintenance of siRNA activity by antisense even after chemical modification may be confirmed by degree of reduction in luciferase exhibited by siRNA.

### Example 7-1

### Preparation of Firefly Luciferase Vector

A sequence complementary to an antisense strand and a sequence complementary to a sense strand of siRNA were respectively cloned in a pMIR-REPORT (Ambion) vector expressing firefly luciferase to prepare two different plasmids. The complementary sequences were designed and synthesized by Cosmo Genetech such that both ends have SpeI and HindIII enzyme site overhang, and then, cloned using SpeI and HindIII enzyme site of a pMIR-REPORT vector.

### Example 7-2

### Measurement of Off-Target Effect of Chemical Lymodified siRNAs

Using plasmids comprising respective sequences complementary to each sense strand and antisense strand of siRNA, prepared in Example 7-1, effects of the antisense and sense strands of siRNA were measured.

Specifically, the firefly luciferase vector prepared in Example 7-1 was transfected in A549 cells (ATCC) together with siRNA, and then, the amount of expressed firefly luciferase was measured by luciferase assay. One day before transfection, A549 cell line was prepared in a 24 well plate at 6*10⁴ cells/well. The luciferase vector (100 ng) in which complementary base sequences were cloned were transfected in Opti-MEM medium (Gibco) using lipofectamine 2000 (Invitrogen) together with a normalizing vector of pRL-SV40 vector (2 ng, Promega) expressing renilla luciferase. After 24 hours, the cells were lyzed using passive lysis buffer, and then, luciferase activity was measured by dual luciferase assay kit (Promega).

The measured firefly luciferase value was normalized for transfection efficiency with the measured renilla luciferase value, and then, percent value to the normalized luciferase value (100%) of control, that was transfected with renilla luciferase vector and firefly luciferase vector in which sequences complementary to each strand were cloned without siRNA, was calculated and described in the following Table 15.

TABLE 15
Sense effect decreased through chemical modification of siRNA

| | | % luciferase activity | |
|---|---|---|---|
| | | | Plasmid comprising |
| | | Plasmid | sequence |
| | | comprising sequence | complementary |
| siRNA | Chemical | complementary to sense | to antisense |
| No. | modification | strand | strand |
| 5 | mod0 | 84.2 | 18.6 |
| | mod1 | 15.6 | 85.7 |
| | mod2 | 67.1 | 42.9 |
| | mod3 | 80.0 | 18.4 |
| | mod4 | 81.7 | 142.7 |
| | mod5 | 29.0 | 40.2 |
| | mod6 | 68.7 | 32.0 |
| | mod7 | 37.3 | 21.1 |
| | mod8 | 73.7 | 40.9 |
| | mod9 | 102.0 | 20.0 |
| | mod10 | 120.1 | 45.1 |
| 9 | mod0 | 51.2 | 4.4 |
| | mod1 | 4.4 | 4.9 |
| | mod2 | 110.7 | 2.0 |
| | mod3 | 55.4 | 98.0 |
| | mod4 | 113.7 | 116.8 |
| | mod5 | 5.9 | 35.9 |
| | mod6 | 96.5 | 4.6 |
| | mod7 | 62.7 | 2.2 |
| | mod8 | 9.1 | 4.3 |
| | mod9 | 72.4 | 13.2 |
| | mod10 | 109.7 | 7.7 |
| 11 | mod0 | 89.9 | 2.9 |
| | mod1 | 85.9 | 12.8 |
| | mod2 | 106.5 | 13.7 |
| | mod3 | 93.7 | 12.7 |
| | mod4 | 74.3 | 26.5 |
| | mod5 | 81.5 | 5.8 |
| | mod6 | 57.4 | 15.5 |
| | mod7 | 55.5 | 6.0 |
| | mod8 | 95.0 | 8.8 |
| | mod9 | 76.2 | 4.8 |
| | mod10 | 79.4 | 5.0 |

(original siRNA that is not chemically modified is indicated by mod0)

As shown in the Table 15, in human lung cancer cell line, unmodified siRNA (mod0) per se had no off-target effect by sense strand in case of siRNA 5 and siRNA 11. However, slight off-target effect by sense strand was seen through decrease in the activity of firefly luciferase having sequence complementary to sense strand of siRNA 9, but if chemically modified, off-target effect was decreased and antisense target effect was maintained, particularly in mod2, 6, 7, 9 and 10.

1. A double stranded siRNA (small interfering RNA) of 15 to 30 bp, which targets an mRNA corresponding to at least one selected from the group consisting of SEQ ID NOs. 2, 3, and 5 to 14 described in the following Table 16.
TABLE 16

| SEQ | |
|---|---|
| ID | |
| NO | Sequence (5′->3′) |
| 2 | GTTTGAACTAACTGGACAC |
| 3 | TGATTTTACTCATCCATGT |
| 5 | GAGAAATGCTTACACACAG |
| 6 | CGAGGAAGAACTATGAACA |
| 7 | GAACATAAAGTCTGCAACA |
| 8 | TGATACCAACAGTAACCAA |
| 9 | TCAGTGTGGGTATAAGAAA |
| 10 | GCTGATTTGTGAACCCATT |
| 11 | GCCGCTCAATTTATGAATA |
| 12 | GCATTGTATGTGTGAATTA |
| 13 | TCAGGATCAGACACCTAGT |
| 14 | ATTTAGACTTGGAGATGTT |

2. The siRNA according to claim 1, wherein the siRNA targets mRNA corresponding to at least one base sequence selected from the group consisting of SEQ ID NOs 6, 10, and 12.

3. The siRNA according to claim 1, wherein the siRNA comprises an overhang consisting of 1 to 5 nucleotides (nt) at 3′ end, 5′ end, or both ends.

4. The siRNA according to claim 1, wherein the siRNA comprises nucleotide sequence selected from the group consisting of siRNA 1, siRNA 2, siRNA 4 to 13, and 18 to 20 described in the following Table 17.
TABLE 17

| SEQ | | | |
|---|---|---|---|
| ID | | | siRNA |
| NO | sequence (5′->3′) | strand | indication |
| 19 | GUUUGAACUAACUGGACACdTdT | Sense | siRNA 1 |
| 20 | GUGUCCAGUUAGUUCAAACdTdT | Antisense | |
| 21 | UGAUUUUACUCAUCCAUGUdTdT | Sense | siRNA 2 |
| 22 | ACAUGGAUGAGUAAAAUCAdTdT | Antisense | |
| 25 | GAGAAAUGCUUACACACAGdTdT | Sense | siRNA 4 |
| 26 | CUGUGUGUAAGCAUUUCUCdTdT | Antisense | |
| 27 | CGAGGAAGAACUAUGAACAdTdT | Sense | siRNA 5 |
| 28 | UGUUCAUAGUUCUUCCUCGdTdT | Antisense | |
| 29 | GAACAUAAAGUCUGCAACAdTdT | Sense | siRNA 6 |
| 30 | UGUUGCAGACUUUAUGUUCdTdT | Antisense | |
| 31 | UGAUACCAACAGUAACCAAdTdT | Sense | siRNA 7 |
| 32 | UUGGUUACUGUUGGUAUCAdTdT | Antisense | |
| 33 | UCAGUGUGGGUAUAAGAAAdTdT | Sense | siRNA 8 |
| 34 | UUUCUUAUACCCACACUGAdTdT | Antisense | |
| 35 | GCUGAUUUGUGAACCCAUUdTdT | Sense | siRNA 9 |
| 36 | AAUGGGUUCACAAAUCAGCdTdT | Antisense | |
| 37 | GCCGCUCAAUUUAUGAAUAdTdT | Sense | siRNA 10 |
| 38 | UAUUCAUAAAUUGAGCGGCdTdT | Antisense | |
| 39 | GCAUUGUAUGUGUGAAUUAdTdT | Sense | siRNA 11 |
| 40 | UAAUUCACACAUACAAUGCdTdT | Antisense | |
| 41 | UCAGGAUCAGACACCUAGUdTdT | Sense | siRNA 12 |
| 42 | ACUAGGUGUCUGAUCCUGAdTdT | Antisense | |
| 43 | AUUUAGACUUGGAGAUGUUdTdT | Sense | siRNA 13 |
| 44 | AACAUCUCCAAGUCUAAAUdTdT | Antisense | |
| 53 | GGAAGAACUAUGAACA | Sense | siRNA 18 |
| 28 | UGUUCAUAGUUCUUCCUCGdTdT | Antisense | |
| 54 | GAUUUGUGAACCCAUU | Sense | siRNA 19 |
| 36 | AAUGGGUUCACAAAUCAGCdTdT | Antisense | |
| 55 | UUGUAUGUGUGAAUUA | Sense | siRNA 20 |
| 40 | UAAUUCACACAUACAAUGCdTdT | Antisense | |

5. The siRNA according to claim 4, wherein the siRNA is selected from the group consisting of
siRNA 5 comprising a sense sequence of SEQ ID NO 27 and an antisense sequence of SEQ ID NO 28,
siRNA 9 comprising a sense sequence of SEQ ID NO 35 and an antisense sequence of SEQ ID NO 36,
siRNA 11 comprising a sense sequence of SEQ ID NO 39 and an antisense sequence of SEQ ID NO 40,
siRNA 18 comprising a sense sequence of SEQ ID NO 53 and an antisense sequence of SEQ ID NO 28,
siRNA 19 comprising a sense sequence of SEQ ID NO 54 and an antisense sequence of SEQ ID NO 36, and
siRNA 20 comprising a sense sequence of SEQ ID NO 55 and an antisense sequence of SEQ ID NO 40.

6. The siRNA according to claim 1, wherein the sugar or base structure of at least one ribonucleic acid, or a bond between the ribonucleic acids is chemically modified.

7. The siRNA according to claim 6, wherein the chemical modification is selected from the group consisting of:
substitution of a phosphodiester bond in the backbone with boranophosphate or phosphorothioate, and
introduction of a methyl group (2′-O-methyl) or a fluoro group (2′-fluoro) at 2′-OH position of a ribose ring.

8. The siRNA according to claim 7, wherein the boranophosphate or phosphorothioate is introduced at 3′ end, 5′ end or both ends.

9. The siRNA according to claim 6, wherein the siRNA comprises nucleotide sequence selected from the group consisting of siRNA 21 to 50 described in the following Table 10.
TABLE 10

| | SEQ | | | | |
|---|---|---|---|---|---|
| | ID | | | | |
| | NO | sequence (5′->3′) | strand | siRNA | Modification |
| Chemically | 56 | CGAGGAAGAACUAUGAACAdT*dT | Sense | siRNA21 | siRNA5- |
| modified | 57 | UGUUCAUAGUUCUUCCUCGdT*dT | Antisense | | mod1 |
| siRNA | 58 | CGAGGAAGAACUAUGAACAdT*dT | Sense | siRNA22 | siRNA5- |
| (30) | 59 | UGUUCAUAGUUCUUCCUCGdT*dT | Antisense | | mod2 |
| | 60 | CGAGGAAGAACUAUGAACAdT*dT | Sense | siRNA23 | siRNA5- |
| | 61 | UGUUCAUAGUUCUUCCUCGdT*dT | Antisense | | mod3 |
| | 62 | CGAGGAAGAACUAUGAACAdT*dT | Sense | siRNA24 | siRNA5- |
| | 63 | UGUUCAUAGUUCUUCCUCGdT*dT | Antisense | | mod4 |
| | 64 | CGAGGAAGAACuAuGAACAdT*dT | Sense | siRNA25 | siRNA5- |
| | 65 | UGuuCAuAGUUCuuCCuCGdT*dT | Antisense | | mod5 |
| | 66 | CGAGGAAGAACUAUGAACAdT*dT | Sense | siRNA26 | siRNA5- |
| | 67 | UGUUCAUAGUUCUUCCUCGdT*dT | Antisense | | mod6 |
| | 68 | CGAGGAAGAACUAUGAACAdT*dT | Sense | siRNA27 | siRNA5- |
| | 69 | UGUUCAUAGUUCUUCCUCGdT*dT | Antisense | | mod7 |
| | 70 | cGAGGAAGAAcuAuGAAcAdT*dT | Sense | siRNA28 | siRNA5- |
| | 71 | uGuucAuAGUcuuccucGdT*dT | Antisense | | mod8 |
| | 72 | CGAGGAAGAACUAUGAACAdT*dT | Sense | siRNA29 | siRNA5- |
| | 73 | UGUUCAUAGUUCUUCCUCGdT*dT | Antisense | | mod9 |
| | 74 | CGAGGAAGAACUAUGAACAdT*dT | Sense | siRNA30 | siRNA5- |
| | 75 | UGUUCAUAGUUCUUCCUCGdT*dT | Antisense | | mod10 |
| | 76 | GCUGAUUUGUGAACCCAUUdT*dT | Sense | siRNA31 | siRNA9- |
| | 77 | AAUGGGUUCACAAAUCAGCdT*dT | Antisense | | mod1 |
| | 78 | GCUGAUUUGUGAACCCAUUdT*dT | Sense | siRNA32 | siRNA9- |
| | 79 | AAUGGGUUCACAAAUCAGCdT*dT | Antisense | | mod2 |
| | 80 | GCUGAUUUGUGAACCCAUUdT*dT | Sense | siRNA33 | siRNA- |
| | 81 | AAUGGGUUCACAAAUCAGCdT*dT | Antisense | | mod3 |
| | 82 | GCUGAUUUGUGAACCCAUUdT*dT | Sense | siRNA34 | siRNA9- |
| | 83 | AAUGGGUUCACAAAUCAGCdT*dT | Antisense | | mod4 |
| | 84 | GCuGAuuuGuGAACCCAuudT*dT | Sense | siRNA35 | siRNA9- |
| | 85 | AAuGGGuuCACAAAuCAGCdT*dT | Antisense | | mod5 |
| | 86 | GCUGAUUUGUGAACCCAUUdT*dT | Sense | siRNA36 | siRNA9- |
| | 87 | AAUGGGUUCACAAAUCAGCdT*dT | Antisense | | mod6 |
| | 88 | GCUGAUUUGUGAACCCAUUdT*dT | Sense | siRNA37 | siRNA9- |
| | 89 | AAUGGGUUCACAAAUCAGCdT*dT | Antisense | | mod7 |
| | 90 | GcuGAuuuGUGAAcccAuudT*dT | Sense | siRNA38 | siRNA9- |
| | 91 | AAuGGGuucACAAAucAGcdT*dT | Antisense | | mod8 |
| | 92 | GCUGAUUUGUGAACCCAUUdT*dT | Sense | siRNA39 | siRNA9- |
| | 93 | AAUGGGUUCACAAAUCAGCdT*dT | Antisense | | mod9 |
| | 94 | GCUGAUUUGUGAACCCAUUdT*dT | Sense | siRNA40 | siRNA9- |
| | 95 | AAUGGGUUCACAAAUCAGCdT*dT | Antisense | | mod10 |
| | 96 | GCAUUGUAUGUGUGAAUUAdT*dT | Sense | siRNA41 | siRNA 11- |
| | 97 | UAAUUCACACAUACAAUGCdT*dT | Antisense | | mod1 |
| | 98 | GCAUUGUAUGUGUGAAUUAdT*dT | Sense | siRNA42 | siRNA 11- |
| | 99 | UAAUUCACACAUACAAUGCdT*dT | Antisense | | mod2 |
| | 100 | GCAUUGUAUGUGUGAAUUAdT*dT | Sense | siRNA43 | siRNA 11- |
| | 101 | UAAUUCACACAUACAAUGCdT*dT | Antisense | | mod3 |
| | 102 | GCAUUGUAUGUGUGAAUUAdT*dT | Sense | siRNA44 | siRNA 11- |
| | 103 | UAAUUCACACAUACAAUGCdT*dT | Antisense | | mod4 |
| | 104 | GCAuuGuAuGuGuGAAuuAdT*dT | Sense | siRNA45 | siRNA 11- |
| | 105 | UAAuuCACACAuACAAuGCdT*dT | Antisense | | mod5 |
| | 106 | GCAUUGUAUGUGUGAAUUAdT*dT | Sense | siRNA46 | siRNA 11- |
| | 107 | UAAUUCACACAUACAAUGCdT*dT | Antisense | | mod6 |
| | 108 | GCAUUGUAUGUGUGAAUUAdT*dT | Sense | siRNA47 | siRNA 11- |
| | 109 | UAAUUCACACAUACAAUGCdT*dT | Antisense | | mod7 |
| | 110 | GcAuuGuAuGuGuGAAuuAdT*dT | Sense | siRNA48 | siRNA 11- |
| | 111 | uAAuucAcACAuAcAAuGcdT*dT | Antisense | | mod8 |
| | 112 | GCAUUGUAUGUGUGAAUUAdT*dT | Sense | siRNA49 | siRNA 11- |
| | 113 | UAAUUCACACAUACAAUGCdT*dT | Antisense | | mod9 |
| | 114 | GCAUUGUAUGUGUGAAUUAdT*dT | Sense | siRNA50 | siRNA 11- |
| | 115 | UAAUUCACACAUACAAUGCdT*dT | Antisense | | mod10 |

in the above Table 10,
notation of chemical modification is as follows:

| notation | Introduced chemical modification |
|---|---|
| * | Phosphodiester bond → phosphorothioate bond |
| underline | 2′-OH → 2′-O—Me |
| Lower case letter | 2′-OH → 2′-F |
| Bold letter | ENA(2′-O, 4′-C ethylene bridged nucleotide) |

the content of modification is as follows, provided that mod1 to mod 7 does not modify 10^{th} and 11^{th} bases of an antisense strand, and dTdT (phosphodiester bond) at 3′ end of all siRNA sense and antisense strands of mod 1 to mod 10 is substituted with a phosphorotioate bond (3′-dT*dT, *: Phosphorothioate bond):

| modification | Chemical modification of siRNA |
|---|---|
| mod1 | 2′-OH group of ribose of 1st and 2nd nucleic acids of antisense strand are |
| | substituted with 2′-O—Me |
| mod2 | in addition to mod1 modification, 2′-OH groups of riboses of 1st and 2nd nucleic |
| | acids of sense strand are substituted with 2′-O—Me |
| mod3 | in addition to mod2 modification, 2′-OH groups of riboses of all U containing |
| | nucleic acids of sense strand are substituted with 2′-O—Me |
| mod4 | in addition to mod3 modification, 2′-OH groups of riboses of all U containing |
| | nucleic acids of antisense strand are substituted with 2′-O—Me) |
| mod5 | in addition to mod1 modification, 2′-OH groups of riboses of all G containing |
| | nucleic acids of sense and antisense strands are substituted with 2′-O—Me, and 2′- |
| | OH groups of riboses of all U containing nucleic acids of sense and antisense |
| | strands are substituted with 2′-F |
| mod6 | in addition to mod1 modification, 5′ end of sense strand is substituted with |
| | ENA(2′-O, 4′-C ethylene bridged nucleotide) |
| mod7 | 2′-OH group of 2^{nd} nucleic acid of 5′ end of antisense strand is substituted with 2′- |
| | O—Me |
| mod8 | 2′-OH groups of all U or C containing nucleic acids of sense and antisense strands |
| | are substituted with 2′-F |
| mod9 | 2′-OH groups of all G containing nucleic acids of sense strand are substituted with |
| | 2′-O—Me, and 2′-OH groups of all nucleic acids containing U of GU sequence, or |
| | 1^{st} U of UUU or UU sequence of antisense strand are substituted with 2′-O—Me |
| mod10 | 2′-OH groups of even-numbered nucleic acids of sense strand are substituted with |
| | 2′-O—Me, and 2′-OH groups of odd-numbered nucleic acids of antisense strand are |
| | substituted with 2′-O—Me |

10. An expression vector comprising the siRNA according to claim 1.

11. The expression vector according to claim 10, wherein the expression vector is selected from the group consisting of plasmid, an adeno-associated virus vector, a retrovirus vector, a vacciniavirus vector, and an oncolytic adenovirus vector.

12. An anticancer composition containing the siRNA according to claim 1 as an active ingredient.

13. The anticancer composition according to claim 12, comprising the siRNA in the form of a complex with a nucleic acid delivery system.

14. The anticancer composition according to claim 13, wherein the nucleic acid delivery system is selected from the group consisting of a viral vector, a non-viral vector, liposome, cationic polymer, micelle, emulsion, and solid lipid nanoparticles.

15. The anticancer composition according to claim 12, further comprising anticancer chemotherapeutics, or siRNA for inhibiting the expression of one selected from the group consisting of growth factor, growth factor receptor, downstream signal transduction protein, viral oncogene, and anticancer agent resistant gene.

16. A method for inhibiting synthesis or expression of Hif1α, comprising
providing a Hif1α-expressing cell separated from the body of an animal; and
contacting the siRNA according to claim 1 with the Hif1α-expressing cells separated from the body.

17. A method for inhibiting growth of cancer cells, comprising
providing a Hif1α-expressing cancer cell separated from the body of an animal; and
contacting the siRNA according to claim 1 with the Hif1α-expressing cancer cells separated from the body.

18. A pharmaceutical composition containing the siRNA according to claim 1 as an active ingredient.


SEQUENCE LISTING (117 sequences)

SEQ ID NO: 1 - DNA, 2481 bp, Artificial Sequence
  sequence of ORF region(cDNA) of human Hif1alpha gene (NM_001530)
atggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactga

SEQ ID NO: 2 - DNA, 19 bp, Artificial Sequence
  siRNA target region on Hif1alpha cDNA
gtttgaactaactggacac

SEQ ID NO: 3 - DNA, 19 bp, Artificial Sequence
  siRNA target region on Hif1alpha cDNA
tgattttactcatccatgt

SEQ ID NO: 4 - DNA, 19 bp, Artificial Sequence
  siRNA target region on Hif1alpha cDNA
catgaggaaatgagagaaa

SEQ ID NO: 5 - DNA, 19 bp, Artificial Sequence
  siRNA target region on Hif1alpha cDNA
gagaaatgcttacacacag

SEQ ID NO: 6 - DNA, 19 bp, Artificial Sequence
  siRNA target region on Hif1alpha cDNA
cgaggaagaactatgaaca

SEQ ID NO: 7 - DNA, 19 bp, Artificial Sequence
  siRNA target region on Hif1alpha cDNA
gaacataaagtctgcaaca

SEQ ID NO: 8 - DNA, 19 bp, Artificial Sequence
  siRNA target region on Hif1alpha cDNA
tgataccaacagtaaccaa

SEQ ID NO: 9 - DNA, 19 bp, Artificial Sequence
  siRNA target region on Hif1alpha cDNA
tcagtgtgggtataagaaa

SEQ ID NO: 10 - DNA, 19 bp, Artificial Sequence
  siRNA target region on Hif1alpha cDNA
gctgatttgtgaacccatt

SEQ ID NO: 11 - DNA, 19 bp, Artificial Sequence
  siRNA target region on Hif1alpha cDNA
gccgctcaatttatgaata

SEQ ID NO: 12 - DNA, 19 bp, Artificial Sequence
  siRNA target region on Hif1alpha cDNA
gcattgtatgtgtgaatta

SEQ ID NO: 13 - DNA, 19 bp, Artificial Sequence
  siRNA target region on Hif1alpha cDNA
tcaggatcagacacctagt

SEQ ID NO: 14 - DNA, 19 bp, Artificial Sequence
  siRNA target region on Hif1alpha cDNA
atttagacttggagatgtt

SEQ ID NO: 15 - DNA, 19 bp, Artificial Sequence
  siRNA target region on Hif1alpha cDNA
agaggtggatatgtctggg

SEQ ID NO: 16 - DNA, 19 bp, Artificial Sequence
  siRNA target region on Hif1alpha cDNA
caccaaagtggaatcagaa

SEQ ID NO: 17 - DNA, 19 bp, Artificial Sequence
  siRNA target region on Hif1alpha cDNA
ttcaagttggaattggtag

SEQ ID NO: 18 - DNA, 21 bp, Artificial Sequence
  siRNA target region on Hif1alpha cDNA
aaagtcggacagcctcaccaa

SEQ ID NO: 19 - RNA, 19 nt, Artificial Sequence
  sense strand of siRNA 1, wherein ′dTdT′ is attached to 3′ end
guuugaacuaacuggacac

SEQ ID NO: 20 - RNA, 19 nt, Artificial Sequence
  antisense strand of siRNA 1, wherein ′dTdT′ is attached to 3′ end
guguccaguuaguucaaac

SEQ ID NO: 21 - RNA, 19 nt, Artificial Sequence
  sense strand of siRNA 2, wherein ′dTdT′ is attached to 3′ end
ugauuuuacucauccaugu

SEQ ID NO: 22 - RNA, 19 nt, Artificial Sequence
  antisense strand of siRNA 2, wherein ′dTdT′ is attached to 3′ end
acauggaugaguaaaauca

SEQ ID NO: 23 - RNA, 19 nt, Artificial Sequence
  sense strand of siRNA 3, wherein ′dTdT′ is attached to 3′ end
caugaggaaaugagagaaa

SEQ ID NO: 24 - RNA, 19 nt, Artificial Sequence
  antisense strand of siRNA 3, wherein ′dTdT′ is attached to 3′ end
uuucucucauuuccucaug

SEQ ID NO: 25 - RNA, 19 nt, Artificial Sequence
  sense strand of siRNA 4, wherein ′dTdT′ is attached to 3′ end
gagaaaugcuuacacacag

SEQ ID NO: 26 - RNA, 19 nt, Artificial Sequence
  antisense strand of siRNA 4, wherein ′dTdT′ is attached to 3′ end
cuguguguaagcauuucuc

SEQ ID NO: 27 - RNA, 19 nt, Artificial Sequence
  sense strand of siRNA 5, wherein ′dTdT′ is attached to 3′ end
cgaggaagaacuaugaaca

SEQ ID NO: 28 - RNA, 19 nt, Artificial Sequence
  antisense strand of siRNA 5, wherein ′dTdT′ is attached to 3′ end
uguucauaguucuuccucg

SEQ ID NO: 29 - RNA, 19 nt, Artificial Sequence
  sense strand of siRNA 6, wherein ′dTdT′ is attached to 3′ end
gaacauaaagucugcaaca

SEQ ID NO: 30 - RNA, 19 nt, Artificial Sequence
  antisense strand of siRNA 6, wherein ′dTdT′ is attached to 3′ end
uguugcagacuuuauguuc

SEQ ID NO: 31 - RNA, 19 nt, Artificial Sequence
  sense strand of siRNA 7, wherein ′dTdT′ is attached to 3′ end
ugauaccaacaguaaccaa

SEQ ID NO: 32 - RNA, 19 nt, Artificial Sequence
  antisense strand of siRNA 7, wherein ′dTdT′ is attached to 3′ end
uugguuacuguugguauca

SEQ ID NO: 33 - RNA, 19 nt, Artificial Sequence
  sense strand of siRNA 8, wherein ′dTdT′ is attached to 3′ end
ucaguguggguauaagaaa

SEQ ID NO: 34 - RNA, 19 nt, Artificial Sequence
  antisense strand of siRNA 8, wherein ′dTdT′ is attached to 3′ end
uuucuuauacccacacuga

SEQ ID NO: 35 - RNA, 19 nt, Artificial Sequence
  sense strand of siRNA 9, wherein ′dTdT′ is attached to 3′ end
gcugauuugugaacccauu

SEQ ID NO: 36 - RNA, 19 nt, Artificial Sequence
  antisense strand of siRNA 9, wherein ′dTdT′ is attached to 3′ end
aauggguucacaaaucagc

SEQ ID NO: 37 - RNA, 19 nt, Artificial Sequence
  sense strand of siRNA 10, wherein ′dTdT′ is attached to 3′ end
gccgcucaauuuaugaaua

SEQ ID NO: 38 - RNA, 19 nt, Artificial Sequence
  antisense strand of siRNA 10, wherein ′dTdT′ is attached to 3′end
uauucauaaauugagcggc

SEQ ID NO: 39 - RNA, 19 nt, Artificial Sequence
  sense strand of siRNA 11, wherein ′dTdT′ is attached to 3′ end
gcauuguaugugugaauua

SEQ ID NO: 40 - RNA, 19 nt, Artificial Sequence
  antisense strand of siRNA 11, wherein ′dTdT′ is attached to 3′end
uaauucacacauacaaugc

SEQ ID NO: 41 - RNA, 19 nt, Artificial Sequence
  sense strand of siRNA 12, wherein ′dTdT′ is attached to 3′ end
ucaggaucagacaccuagu

SEQ ID NO: 42 - RNA, 19 nt, Artificial Sequence
  antisense strand of siRNA 12, wherein ′dTdT′ is attached to 3′ end
acuaggugucugauccuga

SEQ ID NO: 43 - RNA, 19 nt, Artificial Sequence
  sense strand of siRNA 13, wherein ′dTdT′ is attached to 3′ end
auuuagacuuggagauguu

SEQ ID NO: 44 - RNA, 19 nt, Artificial Sequence
  antisense strand of siRNA 13, wherein ′dTdT′ is attached to 3′end
aacaucuccaagucuaaau

SEQ ID NO: 45 - RNA, 19 nt, Artificial Sequence
  sense strand of siRNA 14, wherein ′dTdT′ is attached to 3′ end
agagguggauaugucuggg

SEQ ID NO: 46 - RNA, 19 nt, Artificial Sequence
  antisense strand of siRNA 14, wherein ′dTdT′ is attached to 3′end
cccagacauauccaccucu

SEQ ID NO: 47 - RNA, 19 nt, Artificial Sequence
  sense strand of siRNA 15, wherein ′dTdT′ is attached to 3′ end
caccaaaguggaaucagaa

SEQ ID NO: 48 - RNA, 19 nt, Artificial Sequence
  antisense strand of siRNA 15, wherein ′dTdT′ is attached to 3′end
uucugauuccacuuuggug

SEQ ID NO: 49 - RNA, 19 nt, Artificial Sequence
  sense strand of siRNA 16, wherein ′dTdT′ is attached to 3′ end
uucaaguuggaauugguag

SEQ ID NO: 50 - RNA, 19 nt, Artificial Sequence
  antisense strand of siRNA 16, wherein ′dTdT′ is attached to 3′end
cuaccaauuccaacuugaa

SEQ ID NO: 51 - RNA, 21 nt, Artificial Sequence
  sense strand of siRNA 17
aaagucggacagccucaccaa

SEQ ID NO: 52 - RNA, 21 nt, Artificial Sequence
  antisense strand of siRNA 17
uuggugaggcuguccgacuuu

SEQ ID NO: 53 - RNA, 16 nt, Artificial Sequence
  sense strand of siRNA 18
ggaagaacuaugaaca

SEQ ID NO: 54 - RNA, 16 nt, Artificial Sequence
  sense strand of siRNA 19
gauuugugaacccauu

SEQ ID NO: 55 - RNA, 16 nt, Artificial Sequence
  sense strand of siRNA 20
uuguaugugugaauua

SEQ ID NO: 56 - RNA, 19 nt, Artificial Sequence
  sense strand of siRNA 21, wherein ′dTdT′ is attached to 3′ end, and the ′dTs′ are linked by phosphorothioate bond
cgaggaagaacuaugaaca

SEQ ID NO: 57 - RNA, 19 nt, Artificial Sequence
  antisense strand of siRNA 21, wherein ′dTdT′ is attached to 3′end, the ′dTs′ are linked by phosphorothioate bond, and 2′-OH group of ribose of 1st and 2nd nucleic acids are substituted with 2′-O-Me
uguucauaguucuuccucg

SEQ ID NO: 58 - RNA, 19 nt, Artificial Sequence
  sense strand of siRNA 22, wherein ′dTdT′ is attached to 3′ end, the ′dTs′ are linked by phosphorothioate bond, and 2′-OH groups of ribose of 1st and 2nd nucleic acids are substituted with 2′-O-Me
cgaggaagaacuaugaaca

SEQ ID NO: 59 - RNA, 19 nt, Artificial Sequence
  antisense strand of siRNA 22, wherein ′dTdT′ is attached to 3′end, the ′dTs′ are linked by phosphorothioate bond, and 2′-OH groups of ribose of 1st and 2nd nucleic acids are substituted with 2′-O-Me
uguucauaguucuuccucg

SEQ ID NO: 60 - RNA, 19 nt, Artificial Sequence
  sense strand of siRNA 23, wherein ′dTdT′ is attached to 3′ end, the ′dTs′ are linked by phosphorothioate bond, and 2′-OH groups of ribose of 1st and 2nd nucleic acids and 2′-OH groups of riboses of all U containing nucleic acids are substituted with 2′-O-Me
cgaggaagaacuaugaaca

SEQ ID NO: 61 - RNA, 19 nt, Artificial Sequence
  antisense strand of siRNA 23, wherein ′dTdT′ is attached to 3′end, the ′dTs′ are linked by phosphorothioate bond, and 2′-OH groups of ribose of 1st and 2nd nucleic acids are substituted with 2′-O-Me
uguucauaguucuuccucg

SEQ ID NO: 62 - RNA, 19 nt, Artificial Sequence
  sense strand of siRNA 24, wherein ′dTdT′ is attached to 3′ end, the ′dTs′ are linked by phosphorothioate bond, and 2′-OH groups of ribose of 1st and 2nd nucleic acids and 2′-OH groups of riboses of all U containing nucleic acids are substituted with 2′-O-Me
cgaggaagaacuaugaaca

SEQ ID NO: 63 - RNA, 19 nt, Artificial Sequence
  antisense strand of siRNA 24, wherein ′dTdT′ is attached to 3′ end, the ′dTs′ are linked by phosphorothioate bond, and 2′-OH groups of riboses of 1st and 2nd nucleic acids and all U containing nucleic acids are substituted with 2′-O-Me
uguucauaguucuuccucg

SEQ ID NO: 64 - RNA, 19 nt, Artificial Sequence
  sense strand of siRNA 25, wherein ′dTdT′ is attached to 3′ end, the ′dTs′ are linked by phosphorothioate bond, 2′-OH groups of riboses of all G or U containing nucleic acids are substituted with 2′-O-Me(G case) or 2′-F(U case)
cgaggaagaacuaugaaca

SEQ ID NO: 65 - RNA, 19 nt, Artificial Sequence
  antisense strand of siRNA 25, wherein ′dTdT′ is attached to 3′ end, the ′dTs′ are linked by phosphorothioate bond, 2′-OH groups of riboses of 1st & 2nd n/a and all G or U containing n/a are substituted with 2′-O-Me(1st & 2nd and G case) or 2′-F(U case)
uguucauaguucuuccucg

SEQ ID NO: 66 - RNA, 19 nt, Artificial Sequence
  sense strand of siRNA 26, wherein ′dTdT′ is attached to 3′ end, and the ′dTs′ are linked by phosphorothioate bond, and 5′ end of sense strand is substituted with ENA(2′-O, 4′-C ethylene bridged nucleotide)
cgaggaagaacuaugaaca

SEQ ID NO: 67 - RNA, 19 nt, Artificial Sequence
  antisense strand of siRNA 26, wherein ′dTdT′ is attached to 3′end, the ′dTs′ are linked by phosphorothioate bond, and 2′-OH groups of riboses of 1st and 2nd nucleic acids are substituted with 2′-O-Me
uguucauaguucuuccucg

SEQ ID NO: 68 - RNA, 19 nt, Artificial Sequence
  sense strand of siRNA 27, wherein ′dTdT′ is attached to 3′ end, and the ′dTs′ are linked by phosphorothioate bond
cgaggaagaacuaugaaca

SEQ ID NO: 69 - RNA, 19 nt, Artificial Sequence
  antisense strand of siRNA 27, wherein ′dTdT′ is attached to 3′end, and the ′dTs′ are linked by phosphorothioate bond, and 2′-OH group of 2nd nucleic acid of 5′ end is substituted with 2′-O-Me
uguucauaguucuuccucg

SEQ ID NO: 70 - RNA, 19 nt, Artificial Sequence
  sense strand of siRNA 28, wherein ′dTdT′ is attached to 3′ end, the ′dTs′ are linked by phosphorothioate bond, and 2′-OH groups of all U or C containing nucleic acids are substituted with 2′-F
cgaggaagaacuaugaaca

SEQ ID NO: 71 - RNA, 18 nt, Artificial Sequence
  antisense strand of siRNA 28, wherein ′dTdT′ is attached to 3′ end, the ′dTs′ are linked by phosphorothioate bond, and 2′-OH groups of all U or C containing nucleic acids are substituted with 2′-F
uguucauagucuuccucg

SEQ ID NO: 72 - RNA, 19 nt, Artificial Sequence
  sense strand of siRNA 29, wherein ′dTdT′ is attached to 3′ end, the ′dTs′ are linked by phosphorothioate bond, and 2′-OH groups of all G containing nucleic acids are substituted with 2′-O-Me
cgaggaagaacuaugaaca

SEQ ID NO: 73 - RNA, 19 nt, Artificial Sequence
  antisense strand of siRNA 29, wherein ′dTdT′ is attached to 3′end, the ′dTs′ are linked by phosphorothioate bond, and 2′-OH groups of all nucleic acids containing U of GU, or 1st U of UUU or UU are substituted with 2′-O-Me
uguucauaguucuuccucg

SEQ ID NO: 74 - RNA, 19 nt, Artificial Sequence
  sense strand of siRNA 30, wherein ′dTdT′ is attached to 3′ end, and the ′dTs′ are linked by phosphorothioate bond, and 2′-OH groups of even-numbered nucleic acids are substituted with 2′-O-Me
cgaggaagaacuaugaaca

SEQ ID NO: 75 - RNA, 19 nt, Artificial Sequence
  antisense strand of siRNA 30, wherein ′dTdT′ is attached to 3′end, and the ′dTs′ are linked by phosphorothioate bond, and 2′-OH groups of odd-numbered nucleic acids are substituted with 2′-O-Me
uguucauaguucuuccucg

SEQ ID NO: 76 - RNA, 19 nt, Artificial Sequence
  sense strand of siRNA 31, wherein ′dTdT′ is attached to 3′ end, and the ′dTs′ are linked by phosphorothioate bond
gcugauuugugaacccauu

SEQ ID NO: 77 - RNA, 19 nt, Artificial Sequence
  antisense strand of siRNA 31, wherein ′dTdT′ is attached to 3′end, the ′dTs′ are linked by phosphorothioate bond, and 2′-OH groups of riboses of 1st and 2nd nucleic acids are substituted with 2′-O-Me
aauggguucacaaaucagc

SEQ ID NO: 78 - RNA, 19 nt, Artificial Sequence
  sense strand of siRNA 32, wherein ′dTdT′ is attached to 3′ end, the ′dTs′ are linked by phosphorothioate bond, and 2′-OH groups of riboses of 1st and 2nd nucleic acids are substituted with 2′-O-Me
gcugauuugugaacccauu

SEQ ID NO: 79 - RNA, 19 nt, Artificial Sequence
  antisense strand of siRNA 32, wherein ′dTdT′ is attached to 3′end, the ′dTs′ are linked by phosphorothioate bond, and 2′-OH groups of riboses of 1st and 2nd nucleic acids are substituted  with 2′-O-Me
aauggguucacaaaucagc

SEQ ID NO: 80 - RNA, 19 nt, Artificial Sequence
  sense strand of siRNA 33, wherein ′dTdT′ is attached to 3′ end, the ′dTs′ are linked by phosphorothioate bond, and 2′-OH groups of riboses of 1st and 2nd nucleic acids and 2′-OH groups of riboses of all U containing nucleic acids are substituted with 2′-O-Me
gcugauuugugaacccauu

SEQ ID NO: 81 - RNA, 19 nt, Artificial Sequence
  antisense strand of siRNA 33, wherein ′dTdT′ is attached to 3′end, the ′dTs′ are linked by phosphorothioate bond, and 2′-OH groups of riboses of 1st and 2nd nucleic acids are substituted with 2′-O-Me
aauggguucacaaaucagc

SEQ ID NO: 82 - RNA, 19 nt, Artificial Sequence
  sense strand of siRNA 34, wherein ′dTdT′ is attached to 3′ end, the ′dTs′ are linked by phosphorothioate bond, and 2′-OH groups of ribose of 1st and 2nd nucleic acids and 2′-OH groups of riboses of all U containing nucleic acids are substituted with 2′-O-Me
gcugauuugugaacccauu

SEQ ID NO: 83 - RNA, 19 nt, Artificial Sequence
  antisense strand of siRNA 34, wherein ′dTdT′ is attached to 3′end, the ′dTs′ are linked by phosphorothioate bond, and 2′-OH groups of riboses of 1st and 2nd nucleic acids and all U containing nucleic acids are substituted with 2′-O-Me
aauggguucacaaaucagc

SEQ ID NO: 84 - RNA, 19 nt, Artificial Sequence
  sense strand of siRNA 35, wherein ′dTdT′ is attached to 3′ end, the ′dTs′ are linked by phosphorothioate bond, and 2′-OH groups of riboses of all G or U containing nucleic acids are substituted with 2′-O-Me(G case) or 2′-F(U case)
gcugauuugugaacccauu

SEQ ID NO: 85 - RNA, 19 nt, Artificial Sequence
  antisense strand of siRNA 35, wherein ′dTdT′ is attached to 3′end, the ′dTs′ are linked by phosphorothioate bond, and 2′-OH groups of riboses of 1st & 2nd n/a and all G or U containing n/a are substituted with 2′-O-Me(1st & 2nd and G case) or 2′-F(U case)
aauggguucacaaaucagc

SEQ ID NO: 86 - RNA, 19 nt, Artificial Sequence
  sense strand of siRNA 36, wherein ′dTdT′ is attached to 3′ end, and the ′dTs′ are linked by phosphorothioate bond, and 5′ end of sense strand is substituted with ENA(2′-O, 4′-C ethylene bridged nucleotide)
gcugauuugugaacccauu

SEQ ID NO: 87 - RNA, 19 nt, Artificial Sequence
  antisense strand of siRNA 21, wherein ′dTdT′ is attached to 3′end, the ′dTs′ are linked by phosphorothioate bond, and 2′-OH groups of riboses of 1st and 2nd nucleic acids are substituted with 2′-O-Me
aauggguucacaaaucagc

SEQ ID NO: 88 - RNA, 19 nt, Artificial Sequence
  sense strand of siRNA 37, wherein ′dTdT′ is attached to 3′ end, and the ′dTs′ are linked by phosphorothioate bond
gcugauuugugaacccauu

SEQ ID NO: 89 - RNA, 19 nt, Artificial Sequence
  antisense strand of siRNA 37, wherein ′dTdT′ is attached to 3′ end, and the ′dTs′ are linked by phosphorothioate bond, and 2′-OH group of 2nd nucleic acid of 5′ end is substituted with 2′-O-Me
aauggguucacaaaucagc

SEQ ID NO: 90 - RNA, 19 nt, Artificial Sequence
  sense strand of siRNA 21, wherein ′dTdT′ is attached to 3′ end, the ′dTs′ are linked by phosphorothioate bond, and 2′-OH groups of all U or C containing nucleic acids are substituted with 2′-F
gcugauuugugaacccauu

SEQ ID NO: 91 - RNA, 19 nt, Artificial Sequence
  antisense strand of siRNA 38, wherein ′dTdT′ is attached to 3′ end, the ′dTs′ are linked by phosphorothioate bond, and 2′-OH groups of all U or C containing nucleic acids are substituted with 2′-F
aauggguucacaaaucagc

SEQ ID NO: 92 - RNA, 19 nt, Artificial Sequence
  sense strand of siRNA 39, wherein ′dTdT′ is attached to 3′ end, the ′dTs′ are linked by phosphorothioate bond, and 2′-OH groups of all G containing nucleic acids are substituted with 2′-O-Me
gcugauuugugaacccauu

SEQ ID NO: 93 - RNA, 19 nt, Artificial Sequence
  antisense strand of siRNA 21, wherein ′dTdT′ is attached to 3′ end, the ′dTs′ are linked by phosphorothioate bond, and 2′-OH groups of all nucleic acids containing U of GU, or 1st U of UUU or UU are substituted with 2′-O-Me
aauggguucacaaaucagc

SEQ ID NO: 94 - RNA, 19 nt, Artificial Sequence
  sense strand of siRNA 40, wherein ′dTdT′ is attached to 3′ end, and the ′dTs′ are linked by phosphorothioate bond, and 2′-OH groups of even-numbered nucleic acids are substituted with 2′-O-Me
gcugauuugugaacccauu

SEQ ID NO: 95 - RNA, 19 nt, Artificial Sequence
  antisense strand of siRNA 40, wherein ′dTdT′ is attached to 3′end, and the ′dTs′ are linked by phosphorothioate bond, and 2′-OHgroups of odd-numbered nucleic acids are substituted with 2′-O-Me
aauggguucacaaaucagc

SEQ ID NO: 96 - RNA, 19 nt, Artificial Sequence
  sense strand of siRNA 41, wherein ′dTdT′ is attached to 3′ end, and the ′dTs′ are linked by phosphorothioate bond
gcauuguaugugugaauua

SEQ ID NO: 97 - RNA, 19 nt, Artificial Sequence
  antisense strand of siRNA 21, wherein ′dTdT′ is attached to 3′end, the ′dTs′ are linked by phosphorothioate bond, and 2′-OH groups of riboses of 1st and 2nd nucleic acids are substituted with 2′-O-Me
uaauucacacauacaaugc

SEQ ID NO: 98 - RNA, 19 nt, Artificial Sequence
  sense strand of siRNA 42, wherein ′dTdT′ is attached to 3′ end,  the ′dTs′ are linked by phosphorothioate bond, and 2′-OH groups of riboses of 1st and 2nd nucleic acids are substituted with 2′-O-Me
gcauuguaugugugaauua

SEQ ID NO: 99 - RNA, 19 nt, Artificial Sequence
  antisense strand of siRNA 42, wherein ′dTdT′ is attached to 3′end, the ′dTs′ are linked by phosphorothioate bond, and 2′-OH groups of riboses of 1st and 2nd nucleic acids are substituted with 2′-O-Me
uaauucacacauacaaugc

SEQ ID NO: 100 - RNA, 19 nt, Artificial Sequence
  sense strand of siRNA 43, wherein ′dTdT′ is attached to 3′ end, the ′dTs′ are linked by phosphorothioate bond, and 2′-OH groups of riboses of 1st and 2nd nucleic acids and 2′-OH groups of riboses of all U containing nucleic acids are substituted with 2′-O-Me
gcauuguaugugugaauua

SEQ ID NO: 101 - RNA, 19 nt, Artificial Sequence
  antisense strand of siRNA 43, wherein ′dTdT′ is attached to 3′end, the ′dTs′ are linked by phosphorothioate bond, and 2′-OH groups of riboses of 1st and 2nd nucleic acids are substituted with 2′-O-Me
uaauucacacauacaaugc

SEQ ID NO: 102 - RNA, 19 nt, Artificial Sequence
  sense strand of siRNA 44, wherein ′dTdT′ is attached to 3′ end, the ′dTs′ are linked by phosphorothioate bond, and 2′-OH groups of ribose of 1st and 2nd nucleic acids and 2′-OH groups of riboses of all U containing nucleic acids are substituted with 2′-O-Me
gcauuguaugugugaauua

SEQ ID NO: 103 - RNA, 19 nt, Artificial Sequence
  antisense strand of siRNA 44, wherein ′dTdT′ is attached to 3′end, the ′dTs′ are linked by phosphorothioate bond, and 2′-OH groups of riboses of 1st and 2nd nucleic acids and all U containing nucleic acids are substituted with 2′-O-Me
uaauucacacauacaaugc

SEQ ID NO: 104 - RNA, 19 nt, Artificial Sequence
  sense strand of siRNA 45, wherein ′dTdT′ is attached to 3′ end, the ′dTs′ are linked by phosphorothioate bond, and 2′-OH groups of riboses of all G or U containing nucleic acids are substituted with 2′-O-Me(G case) or 2′-F(U case)
gcauuguaugugugaauua

SEQ ID NO: 105 - RNA, 19 nt, Artificial Sequence
  antisense strand of siRNA 45, wherein ′dTdT′ is attached to 3′end, the ′dTs′ are linked by phosphorothioate bond, and 2′-OH groups of riboses of 1st & 2nd n/a and all G or U containing n/a are substituted with 2′-O-Me(1st & 2nd and G case) or 2′-F(U case)
uaauucacacauacaaugc

SEQ ID NO: 106 - RNA, 19 nt, Artificial Sequence
  sense strand of siRNA 46, wherein ′dTdT′ is attached to 3′ end, and the ′dTs′ are linked by phosphorothioate bond, and 5′ end of sense strand is substituted with ENA(2′-O, 4′-C ethylene bridged nucleotide)
gcauuguaugugugaauua

SEQ ID NO: 107 - RNA, 19 nt, Artificial Sequence
  antisense strand of siRNA 46, wherein ′dTdT′ is attached to 3′end, the ′dTs′ are linked by phosphorothioate bond, and 2′-OH groups of riboses of 1st and 2nd nucleic acids are substituted with 2′-O-Me
uaauucacacauacaaugc

SEQ ID NO: 108 - RNA, 19 nt, Artificial Sequence
  sense strand of siRNA 47, wherein ′dTdT′ is attached to 3′ end, and the ′dTs′ are linked by phosphorothioate bond
gcauuguaugugugaauua

SEQ ID NO: 109 - RNA, 19 nt, Artificial Sequence
  antisense strand of siRNA 47, wherein ′dTdT′ is attached to 3′end, and the ′dTs′ are linked by phosphorothioate bond, and 2′-OH group of 2nd nucleic acid of 5′ end is substituted with 2′-O-Me
uaauucacacauacaaugc

SEQ ID NO: 110 - RNA, 19 nt, Artificial Sequence
  sense strand of siRNA 48, wherein ′dTdT′ is attached to 3′ end, the ′dTs′ are linked by phosphorothioate bond, and 2′-OH groups of all U or C containing nucleic acids are substituted with 2′-F
gcauuguaugugugaauua

SEQ ID NO: 111 - RNA, 19 nt, Artificial Sequence
  antisense strand of siRNA 48, wherein ′dTdT′ is attached to 3′end, the ′dTs′ are linked by phosphorothioate bond, and 2′-OH groups of all U or C containing nucleic acids are substituted with 2′-F
uaauucacacauacaaugc

SEQ ID NO: 112 - RNA, 19 nt, Artificial Sequence
  sense strand of siRNA 49, wherein ′dTdT′ is attached to 3′ end, the ′dTs′ are linked by phosphorothioate bond, and 2′-OH groups of all G containing nucleic acids are substituted with 2′-O-Me
gcauuguaugugugaauua

SEQ ID NO: 113 - RNA, 19 nt, Artificial Sequence
  antisense strand of siRNA 49, wherein ′dTdT′ is attached to 3′end, the ′dTs′ are linked by phosphorothioate bond, and 2′-OH groups of all nucleic acids containing U of GU, or 1st U of UUU or UU are substituted with 2′-O-Me
uaauucacacauacaaugc

SEQ ID NO: 114 - RNA, 19 nt, Artificial Sequence
  sense strand of siRNA 50, wherein ′dTdT′ is attached to 3′ end, and the ′dTs′ are linked by phosphorothioate bond, and 2′-OH groups of even-numbered nucleic acids are substituted with 2′-O-Me
gcauuguaugugugaauua

SEQ ID NO: 115 - RNA, 19 nt, Artificial Sequence
  antisense strand of siRNA 50, wherein ′dTdT′ is attached to 3′end, and the ′dTs′ are linked by phosphorothioate bond, and 2′-OH groups of odd-numbered nucleic acids are substituted with 2′-O-Me
uaauucacacauacaaugc

SEQ ID NO: 116 - RNA, 21 nt, Artificial Sequence
  sense strand of siApoB-1 siRNA
gucaucacacugaauaccaau

SEQ ID NO: 117 - RNA, 23 nt, Artificial Sequence
  antisense strand of siApoB-1 siRNA
auugguauucagugugaugacac